(19) United States
(12) Patent Application Publication
(10) Pub. No.: US 2007/0031492 A1
(43) Pub. Date: Feb. 8, 2007

(54) Remote controlled synthesis system

(76) Inventors: Douglas D. Dischino, Middlefield, CT (US); Christopher J. Bernard, Cheshire, CT (US); James J. Mongillo, Northford, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/495,109

(22) Filed: Jul. 28, 2006

Related U.S. Application Data
(60) Provisional application No. 60/704,686, filed on Aug. 2, 2005.

(51) Int. Cl. A61K 9/22

(52) U.S. Cl. 424/468

(57) ABSTRACT

A synthesis system and a method for implementing the synthesis system is provided and includes a first synthesis portion and a second synthesis portion. The first synthesis portion includes at least one input device, at least one material collection device, at least one container and at least one configurable flow direction device. The second synthesis portion includes a support platform and at least one substance processing device structure, wherein at least one of the support platform and the at least one substance processing device structure is configurable to dispose the at least one substance processing device structure adjacent the at least one container.

### CROSS REFERENCE TO RELATED PATENT APPLICATIONS

This application claims the benefit of U.S. Provisional Patent Application No. 60/704,686, filed Aug. 2, 2005.

### FIELD OF THE INVENTION

This disclosure relates generally to an apparatus for the handling and processing of materials and more particularly to a remotely controlled system for handling and processing materials.

### BACKGROUND OF THE INVENTION

As pharmaceutical developments in nuclear medicine and disease diagnostic techniques advance and improve, the advantages that nuclear medicine has over conventional medical techniques for certain applications are becoming more apparent. As such, the use of radioactive substances, such as radionuclides, for the detection and diagnosis of diseases, such as detecting tumors, irregular/inadequate blood flow to various tissues and inadequate functioning of organs has increased in popularity. To date, a variety of applications using radionuclides exist and include nuclear imaging techniques that are far superior to conventional imaging techniques, such as Positron Emission Tomography (PET), Single Photon Emission Computed Tomography (SPECT), Cardiovascular Imaging and Bone Scanning.

For example, Positron Emission Tomography (PET) is a high resolution, non-invasive, imaging technique which uses the decaying properties of a radionuclide to visualize disease in living tissue. As such, PET imaging is a valuable tool for studying subjects, such as primates, for the development of pharmaceuticals to treat a variety of health conditions. During the PET procedure, a radionuclide is used to produce a plurality of radioactive particles for detection by the PET device. A radionuclide is an unstable substance which emits subatomic particles (e.g. beta particles, alpha particles, neutrons, positrons and/or photons) as it decays, wherein the type of subatomic particles emitted is dependent upon the type of radionuclide. For example, fluorine-18 (¹⁸F), which emits β+ particles and has a half-life (t ½) of 110 minutes, is one of the most widely used positron-emitting nuclides in a clinical setting. As the ¹⁸F decays a positively charged electron, called a positron, is emitted from the nucleus with a kinetic energy of several hundred KeV. Each positron then travels a finite distance before interacting with an electron from a different atom to form a transient species called a positronium ion. The positronium ion then undergoes annihilation producing two photons, or gamma rays, each of which have an energy equal to 511 keV and a nearly opposing direction of motion (180° from each other). These photons, or gamma rays, are detected by a ring of detectors (scintillators) that encircle the subject that is being imaged. Because each annihilation event creates two 511 keV photons traveling in opposite directions, coincidence detection circuits record only those photons that are detected simultaneously by two detectors located on opposing sides of the subject. The number of such simultaneous events indicates the number of positron annihilation events that occurred along a line joining the two opposing detectors. Typically, within a few minutes, hundreds of millions of events are recorded to indicate the number of annihilation events along lines joining pairs of detectors in the ring. These numbers are then used to create a high resolution image using well known tomography techniques.

However, several problems currently exist with working with radioactive materials, such as ¹⁸F or 99^{m}Tc-Cardolite. One problem involves the radiation exposure received by the scientists working with these materials. Unlike patients who may only be exposed to a source of radiation infrequently throughout their lifetime, those individuals who receive daily exposure to radiation, such as a radiochemist and/or a radiopharmacist who process these materials, are at a far greater risk for health problems. This is because these substances emit an ionizing radiation, as briefly discussed hereinabove. As such, when this radiation interacts with the atoms of a living subject, orbital electrons surrounding these atoms can be ‘knock’ off by the collisions with the emitted particles. It is well known that the ‘loss’ of an electron from atoms in living tissue can cause health and development problems for that tissue ranging from cell death to genetic mutation leading to birth defects and/or cancer. Thus, the only known way to work with these substances and avoid health consequences is to eliminate or reduce exposure of the radiochemist to the ionizing radiation. In fact, the actions of those involved in the routine handling of radioactive materials are guided by the ALARA recommendation of the Nuclear Regulatory Commission which states that at all times exposure to radioactive material should be As Low As Reasonably Achievable. One way to reduce exposure is by working with these substances while they are disposed in containers shielded with lead. For example, radiation emitted from ¹⁸F requires a lead shield of approximate two inches in width to stop the emitted radiation. Another way to reduce exposure is to reduce the amount of “hands on” interaction by the radiochemist required during the processing of these substances.

Unfortunately, current methods and devices for processing these radioactive materials typically require handling of the radioactive materials at each step in the process. Thus, because the radioactive material must still be handled and prepared at each step radiation exposure to the radiochemist is not minimized. Historically only large medical centers, universities or national laboratories equipped with a cyclotron to produce the positron-emitting radioisotope and PET cameras were involved in the synthesis and utilization of these short lived radionuclides. In these situations, the ¹⁸F produced in the cyclotron target would be transferred via tubing directly into a hot cell where the radiosynthesis of compounds would occur. Following high performance liquid chromatography purification and subsequent formulation, this material would then be available for clinical studies. Recently however, there has been the advent of cyclotron-free PET imaging centers. This has been made possible by the creation of regional production facilities which are responsible for the synthesis, purification and distribution of ¹⁸F labeled compounds, primarily ¹⁸F-FDG, fluorodeoxyglucose. These facilities arrange for land transportation of the radiolabeled product suitable for human use to cyclotron-free PET imaging centers, which can be as far as 100-150 miles from the production facility.

Using the same model, cyclotron-free radiosynthesis facilities have been created in private industry for the purpose of preparing proprietary radiolabeled compounds for drug discovery and development operations. In this situation, one scenario is as follows. The aqueous ¹⁸F is obtained directly from the cyclotron target and may be disposed within a glass vial. The glass vial containing the aqueous ¹⁸F is then shipped to a user of the material via a lead shipping container or pig. Upon receipt of the radioactive material, the glass vial containing the radioactive material is removed from the shipping pig and inserted into a second pig which is then introduced into the hot cell. The vial is then connected to the synthesis system by an assembly of needles connected to tefzel tubing and the first reaction stage is initiated by forcing the radioactive material out of the second needle via the addition of nitrogen gas to the vial. It should be appreciated that at each stage of the synthesis process, current methods and devices require that the radioactive material be handled by the radiochemist. This is undesirable because each time the radioactive material is handled the material handler is exposed to radiation.

Although steps are taken to shield the radiochemist in order to reduce the overall exposure to radiation, certain body parts still experience a higher than desired level of exposure. Specifically, the fingers and hands of the radiochemist still experience a higher than desired level of exposure because different processes require that the radioactive material be transferred from one container to another. One reason is because the glass vial used to transport the radioactive material typically includes a screw on/screw off cap which must be manually removed by the radiochemist by gripping the glass vial with one hand and removing the cap with the other hand. Because the hands of the radiochemist must be unprotected to allow the hands of the radiochemist to have a full range of movement during the vial gripping and vial cap removal process, the hands of the radiochemist is exposed to an undesired dose of radiation.

Another problem that exists when working with radioactive materials contained within glass vials involves the possible breakage of a vial containing radioactive material. For example, if a glass vial is broken during the process of removing of the vial cap, the radiochemist may cut open his/her hand on the broken glass and the radioactive material may spill out of the vial causing, not only an environmental exposure to the radioactive material, but also the possible introduction of the radioactive material into the radiochemist via the wound. Reducing the amount of handling required by the radiochemist during the synthesis process would aid in reducing any possible unwanted human exposure radiation and/or to the radioactive material.

### SUMMARY OF THE INVENTION

A synthesis system is provided, wherein the synthesis system includes a first synthesis portion, wherein the first synthesis portion includes, a first station, wherein the first station includes an input needle communicated with a first Sep-Pak device via a first flow tube, wherein the first Sep-Pak device is connected to at least one configurable flow direction device which is further communicated with a first needle, the first needle being configurable to be at least partially disposed within a first vial cavity defined by a first vial, the first station further including a second needle, wherein the second needle is configurable to be at least partially disposed within the first vial cavity, a second station, wherein the second station includes a second vial defining a second vial cavity, a third needle and a fourth needle, wherein the third needle and the fourth needle are configurable to be at least partially disposed within the second vial cavity, a third station, wherein the third station includes a third vial defining a third vial cavity, wherein the third vial cavity is communicated with the second needle and a second Sep-Pak device, wherein the second Sep-Pak device is further communicated with at least one of a first syringe device and a fourth vial cavity defined by a fourth vial via the at least one configurable flow direction device, a fourth station, wherein the fourth station includes a fifth needle configurable to be at least partially disposed within the fourth vial cavity, wherein the fifth needle is communicated with at least one of a second syringe device and an HPLC loop via the at least one configurable flow direction device, and an HPLC station, wherein the HPLC station includes an HPLC column includes an HPLC input port and an HPLC output port, the HPLC input port communicated with the HPLC loop and the HPLC output port communicated with the at least one configurable flow device and a second synthesis portion, wherein the second synthesis portion includes, a support platform and at least one device structure, wherein the support platform is configurable to dispose the at least one device structure adjacent at least one of the first vial, the second vial and the third vial and wherein the at least one device structure is configurable to be associated with at least one of the first vial, the second vial and the third vial.

A synthesis system is provided and includes at least one synthesis portion, wherein the at least one synthesis portion includes at least one synthesis portion input device and at least one processing portion, wherein the at least one processing portion includes at least one processing portion input device. The synthesis system also includes at least one support device for securably supporting a container, wherein the at least one support device is configurable to support a plurality of different sized and shaped containers and wherein the at least one support device is communicated with at least one of the at least one synthesis portion and the at least one processing portion via at least one configurable flow valve.

A synthesis system is provided and includes a first synthesis portion, wherein the first synthesis portion includes, at least one input device, at least one material collection device, at least one container and at least one configurable flow direction device and a second synthesis portion, wherein the second synthesis portion includes, a support platform and at least one substance processing device structure, wherein at least one of the support platform and the at least one substance processing device structure is configurable to dispose the at least one substance processing device structure adjacent the at least one container.

A method for implementing a synthesis system having a first synthesis portion and a second synthesis portion is provided, wherein the first synthesis portion includes a plurality of synthesis stations having at least one input device, at least one output device and at least one container defining a container cavity, wherein each of the at least one input and the at least one output is configurably communicated with the container cavity and wherein the second synthesis portion includes a support platform and at least one substance processing device structure, wherein at least one of the support platform and the at least one substance processing device structure is configurable to dispose the at least one substance processing device structure adjacent at least one of the plurality of synthesis stations wherein the at least one substance processing device structure is configurable to be associated with the at least one container. The method includes arranging at least one of the plurality of synthesis stations in a predetermined configuration, wherein the predetermined configuration is responsive to an initial substance to be processed, introducing the initial substance into the at least one of the plurality of synthesis stations via the at least one input, operating the synthesis system to process the initial substance responsive to a predetermined algorithm to generate a processed substance and collecting the processed substance.

A machine-readable computer program code is also provided, wherein the program code includes instructions for causing a controller to implement a method for implementing a synthesis system having a first synthesis portion and a second synthesis portion, wherein the first synthesis portion includes a plurality of synthesis stations having at least one input device, at least one output device and at least one container defining a container cavity, wherein each of the at least one input and the at least one output is configurably communicated with the container cavity and wherein the second synthesis portion includes a support platform and at least one substance processing device structure, wherein at least one of the support platform and the at least one substance processing device structure is configurable to dispose the at least one substance processing device structure adjacent at least one of the plurality of synthesis stations wherein the at least one substance processing device structure is configurable to be associated with the at least one container. The method includes arranging at least one of the plurality of synthesis stations in a predetermined configuration, wherein the predetermined configuration is responsive to an initial substance to be processed, introducing the initial substance into the at least one of the plurality of synthesis stations via the at least one input, operating the synthesis system to process the initial substance responsive to a predetermined algorithm to generate a processed substance and collecting the processed substance.

### BRIEF DESCRIPTION OF DRAWINGS

The foregoing and other features and advantages of the present invention will be more fully understood from the following detailed description of illustrative embodiments, taken in conjunction with the accompanying drawings in which like elements are numbered alike:

FIG. 1 is a front view of a Remotely Controlled Synthesis Device disposed outside of its shielded enclosure, in accordance with an exemplary embodiment;

FIG. 2 is a front view of the Remotely Controlled Synthesis Device of FIG. 1 configured for the synthesis of an ¹⁸F compound;

FIG. 3 is a front view of the first synthesis station of the Remotely Controlled Synthesis Device of FIG. 2;

FIG. 4 is a front view of the first synthesis station of the Remotely Controlled Synthesis Device of FIG. 2;

FIG. 5 is a front view of the first synthesis station of the Remotely Controlled Synthesis Device of FIG. 2;

FIG. 6 is a front view of the first configurable flow direction device used in the Remotely Controlled Synthesis Device of FIG. 2;

FIG. 7 is a front view of the second configurable flow direction device used in the Remotely Controlled Synthesis Device of FIG. 2;

FIG. 8 is a front view of the second synthesis station of the Remotely Controlled Synthesis Device of FIG. 2;

FIG. 9 is a front view of the second synthesis station of the Remotely Controlled Synthesis Device of FIG. 2;

FIG. 10 is a front view of the second synthesis station of the Remotely Controlled Synthesis Device of FIG. 2;

FIG. 11 is a front view of the third synthesis station of the Remotely Controlled Synthesis Device of FIG. 2;

FIG. 12 is a front view of the third synthesis station of the Remotely Controlled Synthesis Device of FIG. 2;

FIG. 13 is a front view of the fourth synthesis station of the Remotely Controlled Synthesis Device of FIG. 2;

FIG. 14 is a front view of the fourth synthesis station of the Remotely Controlled Synthesis Device of FIG. 2;

FIG. 15 is a front view of the fourth synthesis station of the Remotely Controlled Synthesis Device of FIG. 2;

FIG. 16 is a front view of the fourth synthesis station of the Remotely Controlled Synthesis Device of FIG. 2;

FIG. 17 is a front view of the second synthesis portion for the Remotely Controlled Synthesis Device of FIG. 2;

FIG. 18 is a side sectional view of the second synthesis portion for the Remotely Controlled Synthesis Device of FIG. 2;

FIG. 19 is a front view of the second synthesis portion for the Remotely Controlled Synthesis Device of FIG. 2;

FIG. 20 is a side sectional view of the second synthesis portion for the Remotely Controlled Synthesis Device of FIG. 2;

FIG. 21 is a front view of the shielded enclosure for the Remotely Controlled Synthesis Device of FIG. 2;

FIG. 22 is a front view of the shielded enclosure for the Remotely Controlled Synthesis Device of FIG. 2;

FIG. 23 is a right side view of the shielded enclosure for the Remotely Controlled Synthesis Device of FIG. 2;

FIG. 24 is a right side view of the shielded enclosure for the Remotely Controlled Synthesis Device of FIG. 2;

FIG. 25 is a left side view of the shielded enclosure for the Remotely Controlled Synthesis Device of FIG. 2;

FIG. 26 is a left side view of the shielded enclosure for the Remotely Controlled Synthesis Device of FIG. 2;

FIG. 27 is a block diagram describing a method for implementing the Remotely Controlled Synthesis Device of FIG. 2;

FIG. 28 is a front view of the Remotely Controlled Synthesis Device of FIG. 2 configured for the synthesis of an ¹⁸F compound;

FIG. 29 is a front view of the Remotely Controlled Synthesis Device of FIG. 2 configured for the synthesis of an ¹⁸F compound;

FIG. 30 is a front view of the Remotely Controlled Synthesis Device of FIG. 2 configured for the synthesis of an ¹⁸F compound;

FIG. 31 is a front view of the Remotely Controlled Synthesis Device of FIG. 2 configured for the synthesis of an ¹⁸F compound;

FIG. 32 is a front view of the Remotely Controlled Synthesis Device of FIG. 2 configured for the synthesis of an ¹⁸F compound;

FIG. 33 is a front view of the Remotely Controlled Synthesis Device of FIG. 2 configured for the synthesis of an ¹⁸F compound;

FIG. 34 is a front view of the Remotely Controlled Synthesis Device of FIG. 2 configured for the synthesis of an ¹⁸F compound;

FIG. 35 is a front view of the Remotely Controlled Synthesis Device of FIG. 2 configured for the synthesis of an ¹⁸F compound;

FIG. 36 is a front view of the Remotely Controlled Synthesis Device of FIG. 2 configured for the synthesis of an ¹⁸F compound;

FIG. 37 is a front view of the Remotely Controlled Synthesis Device of FIG. 2 configured for the synthesis of an ¹⁸F compound;

FIG. 38 is a front view of the Remotely Controlled Synthesis Device of FIG. 2 configured for the synthesis of an ¹⁸F compound;

FIG. 39 is a front view of the Remotely Controlled Synthesis Device of FIG. 2 configured for the synthesis of an ¹⁸F compound;

FIG. 40 is a front view of the Remotely Controlled Synthesis Device of FIG. 2 configured for the synthesis of an ¹⁸F compound;

FIG. 41 is a front view of the Remotely Controlled Synthesis Device of FIG. 2 configured for the synthesis of an ¹⁸F compound;

FIG. 42 is a front view of the Remotely Controlled Synthesis Device of FIG. 2 configured for the synthesis of an ¹⁸F compound;

FIG. 43 is a front view of the Remotely Controlled Synthesis Device of FIG. 2 configured for the synthesis of an ¹⁸F compound;

FIG. 44 is a front view of the Remotely Controlled Synthesis Device of FIG. 2 configured for the synthesis of an ¹⁸F compound;

FIG. 45 is a front view of the Remotely Controlled Synthesis Device of FIG. 2 configured for the synthesis of an ¹⁸F compound;

FIG. 46 is a front view of the Remotely Controlled Synthesis Device of FIG. 2 configured for the synthesis of an ¹⁸F compound;

FIG. 47 is a front view of the Remotely Controlled Synthesis Device of FIG. 2 configured for the synthesis of an ¹⁸F compound;

FIG. 48 is a front view of the Remotely Controlled Synthesis Device of FIG. 2 configured for the synthesis of an ¹⁸F compound;

FIG. 49 is a front view of a container holding device being automatically transferred between a cyclotron and/or a shielded enclosure and the Remotely Controlled Synthesis Device of FIG. 2;

FIG. 50 is a front view of a container holding device being automatically transferred between a cyclotron and/or a shielded enclosure and the Remotely Controlled Synthesis Device of FIG. 2;

FIG. 51 is a block schematic diagram of a general purpose computing system for implementing the method of FIG. 27;

FIG. 52 is a screen shot of a first embodiment of a software Graphical User Interface used to operate the Remotely Controlled Synthesis Device of FIG. 2 via the general purpose computing system of FIG. 51; and

FIG. 53 is a screen shot of a second embodiment of a software Graphical User Interface used to operate the Remotely Controlled Synthesis Device of FIG. 2 via the general purpose computing device of FIG. 51.

### DETAILED DESCRIPTION OF THE INVENTION

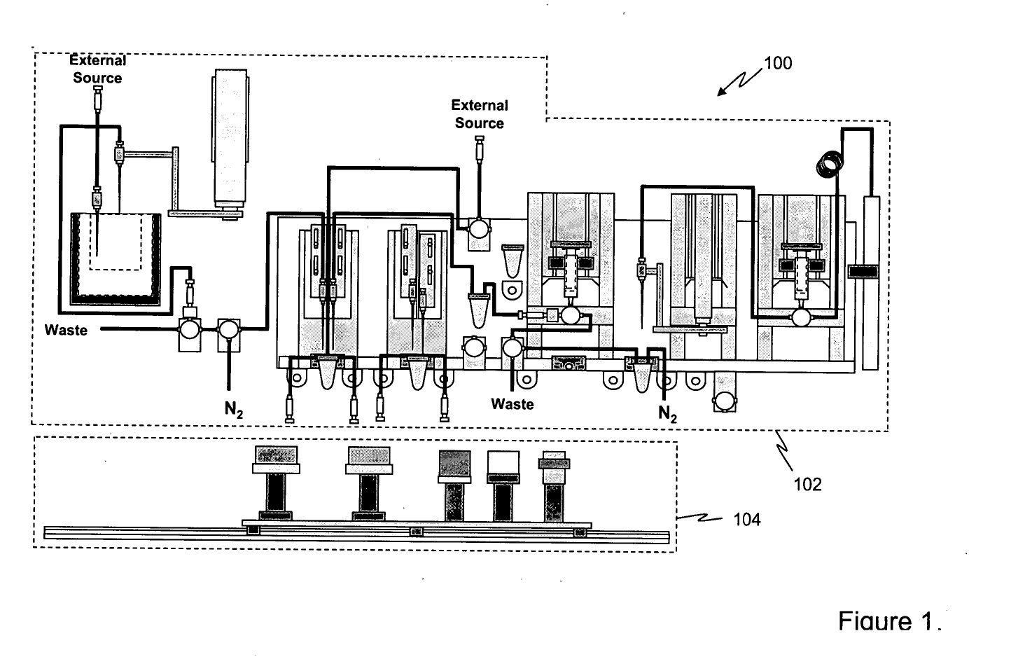

Referring to FIG. 1, an overall embodiment of a remotely controlled synthesis device (RCSD) 100 is shown and includes a first synthesis portion 102 and a second synthesis portion 104.

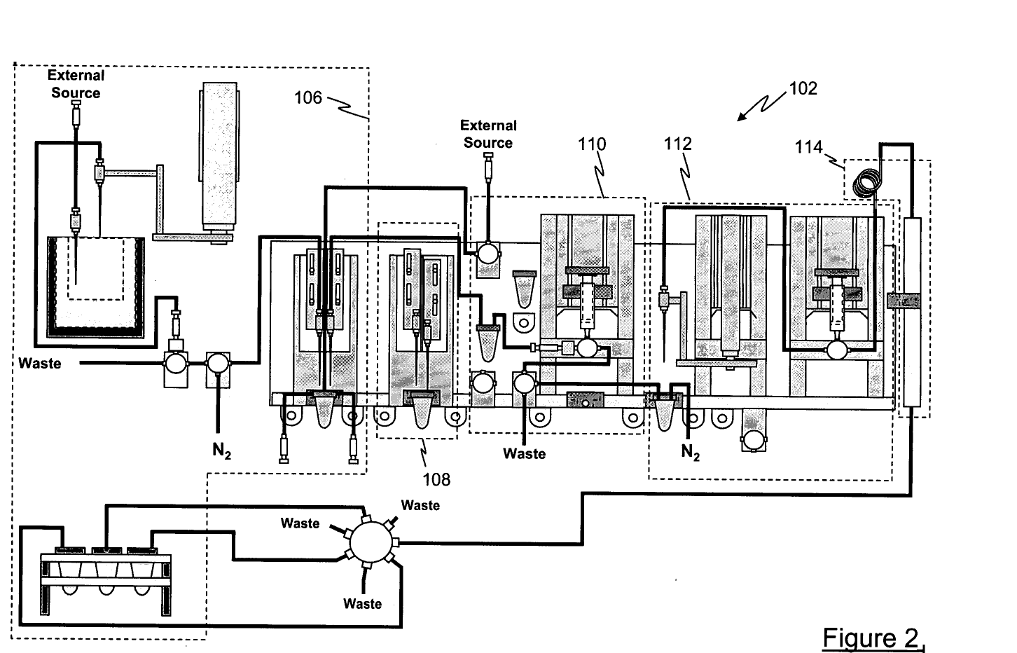

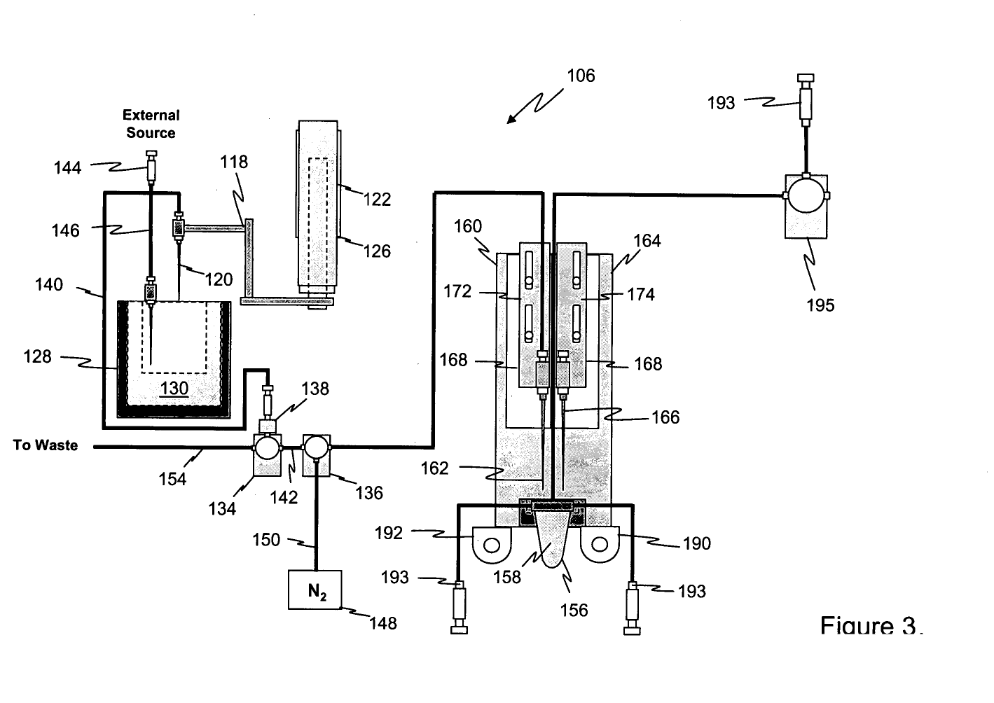

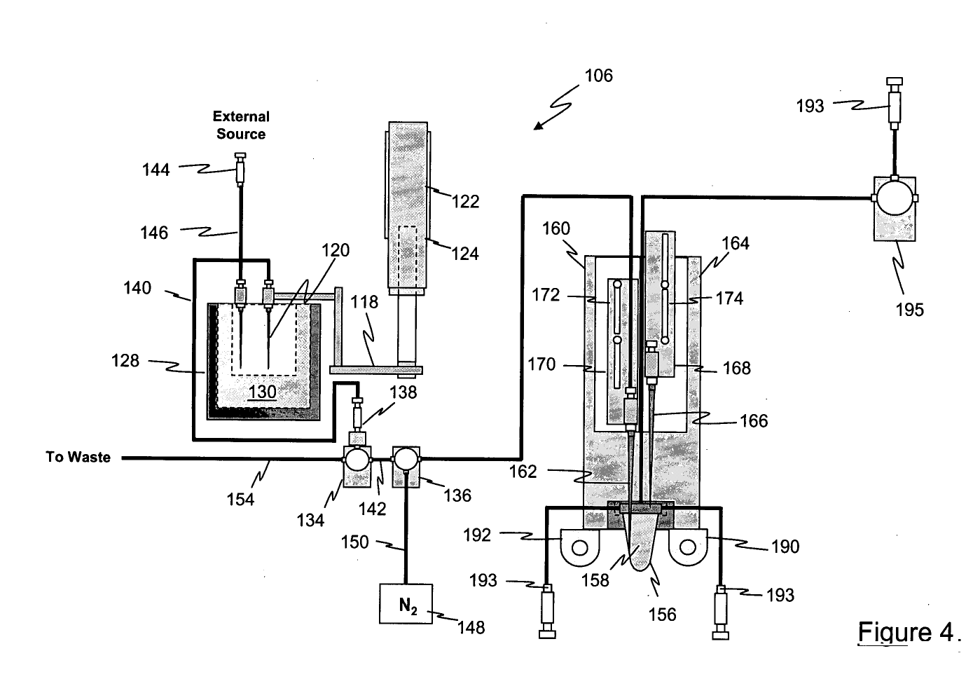

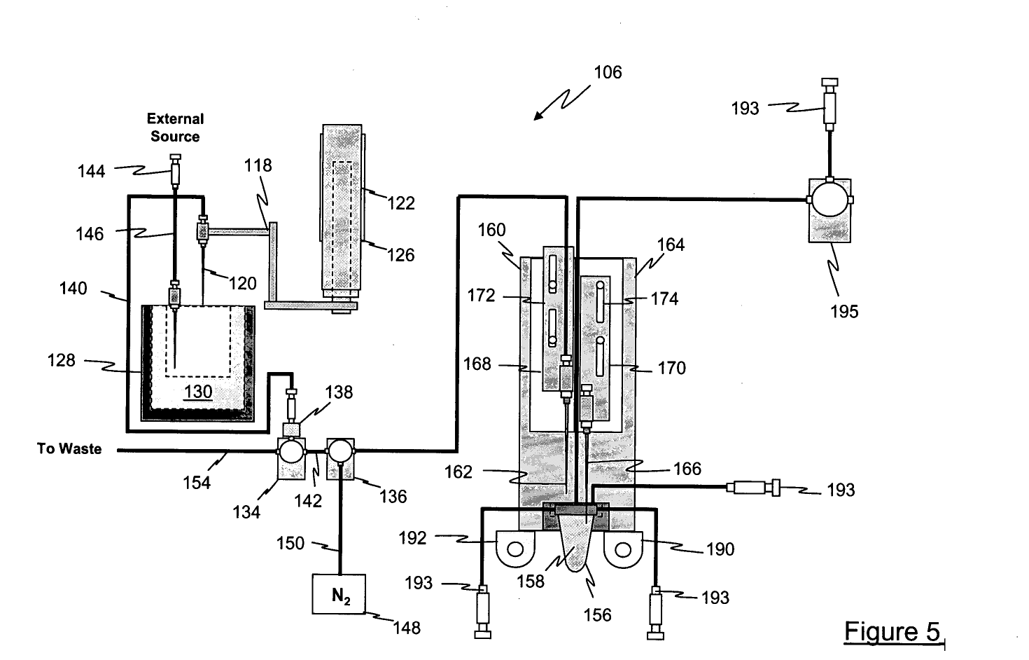

Referring to FIG. 2, the first synthesis portion 102 includes a first synthesis station 106, a second synthesis station 108, a third synthesis station 110, a fourth synthesis station 112 and a High Pressure Liquid Chromatography (HPLC) station 114. As shown in FIG. 3, FIG. 4 and FIG. 5, the first synthesis station 106 includes an input needle device 118 having an input needle 120 and an input needle actuation device 122, wherein the input needle device 118 is connected to the input needle actuation device 122 to allow the input needle device 118 to be configurable between an engaged configuration 124 and a disengaged configuration 126. The input station 106 further includes a container holding device 128 which defines a container holding device cavity 130, wherein the container holding device 128 is disposed such that when the input needle device 118 is configured into the engaged configuration 124, the input needle 120 is disposed within the container holding device cavity 130 and wherein the container holding device 128 is disposed such that when the input needle device 118 is configured into the disengaged configuration 126, the input needle 120 is disposed away from the container holding device cavity 130.

Input station 106 further includes a first configurable flow direction device 134 and a second configurable flow direction device 136, wherein the input needle 120 is connected to the first configurable flow direction device 134 via a first Sep-Pak device 138 and a first flow tube 140 and wherein the first configurable flow direction device 134 is further connected to the second configurable flow direction device 136 via a second flow tube 142. Additionally, the input station 106 may further include a first external input device 144 communicated with the container holding device cavity 130 via a first external input tube 146 for introducing a substance, such as a reagent, to the product contained within the container holding device cavity 130. Additionally, a first N₂ supply device 148 may be connected to the second configurable flow direction device 136 via a first N₂ supply tube 150. Additionally, other devices suitable to the desired end purpose may be connected to the first configurable flow direction device 134 as desired, such as a vacuum device and/or a waste container.

The first synthesis station 106 also includes a first vial 156 defining a first vial cavity 158, a first needle device 160 having a first needle 162 and a second needle device 164 having a second needle 166, wherein each of the first needle device 160 and the second needle device 164 are separately configurable between a disengaged configuration 168 and an engaged configuration 170 via a first needle actuation device 172 and a second needle actuation device 174, respectively. It should be appreciated that the first vial 156 may be disposed to be associated with the first needle device 160 such that when the first needle device 160 is configured into the disengaged configuration 168, the first needle 162 is disposed away from the first vial cavity 158 and when the first needle device 160 is configured into the engaged configuration 170, the first needle 162 is at least partially disposed within the first vial cavity 158. In a similar fashion, when the second needle device 164 is configured into the disengaged configuration 168, the second needle 166 is disposed away from the first vial cavity 158 and when the second needle device 164 is configured into the engaged configuration 170, the second needle 166 is at least partially disposed within the first vial cavity 158.

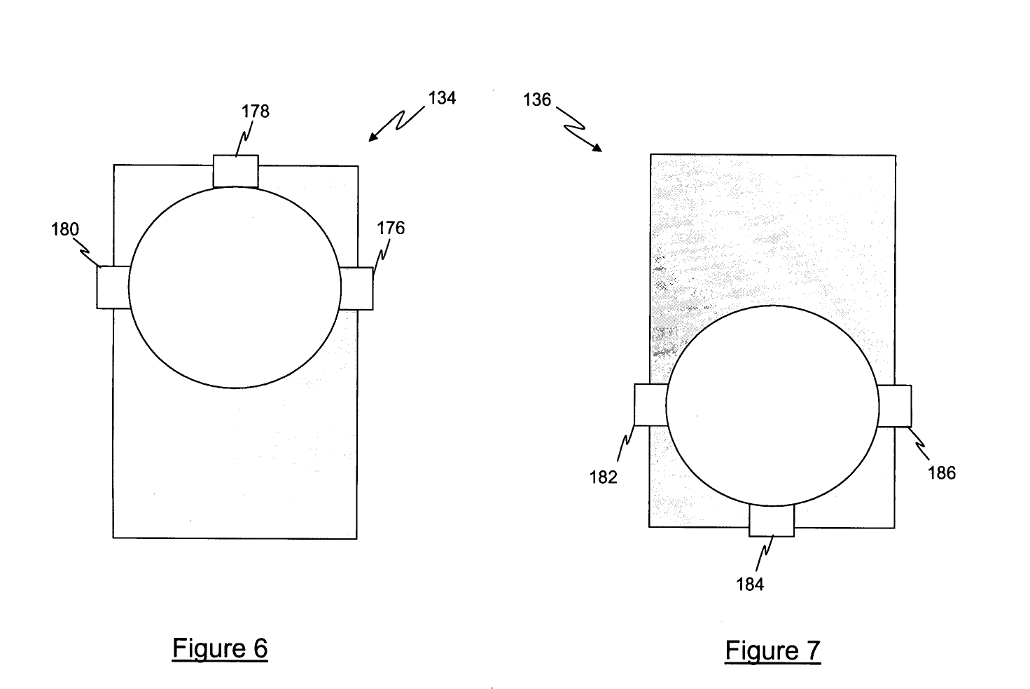

Referring to FIG. 6 and FIG. 7, the first configurable flow direction device 134 includes a first valve first port 176, a first valve second port 178 and a first valve third port 180 and the second configurable flow direction device 136 includes a second valve first port 182, a second valve second port 184 and a second valve third port 186. As can be seen by referring back to FIG. 3, the first Sep-Pak device 138 is connected to the first valve second port 178 and the input needle device 118 such that any fluid flowing between the first configurable flow direction device 134 and the input needle 120 via the first flow tube 140 must flow through the first Sep-Pak device 138. Moreover, the first valve first port 176 is connected to the second valve first port 182 via the second flow tube 142 and the second valve third port 186 is connected to the first needle 162 via a third flow tube 188. Additionally, the first valve third port 180 and/or the second valve second port 184 may be connected to additional devices, such as a vacuum device, a waste container and/or the N₂ supply device 148.

The first configurable flow direction device 134 may be controllably configured such that at least two of the first valve first port 176, the first valve second port 178 and the first valve third port 180 are communicated with each other and the second configurable flow direction device 136 may be controllably configured such that at least two of the second valve first port 182, the second valve second port 184 and the second valve third port 186 are communicated with each other. As such, the first configurable flow direction device 134 and the second configurable flow direction device 136 may be controllably configured to communicate the input needle 120 with the first needle 162 via the first vial 156. Referring back to FIG. 5, the first synthesis station 106 may also include a first temperature sensing device 190 and a first radiation monitoring device 192, wherein the first temperature sensing device 190 may be disposed to sense the temperature of the first vial 156 and the first radiation monitoring device 192 may be movably configurable to be disposed to sense any radiation that may be emitted from at least one of the first Sep-Pak device 138 and the first vial 156. Furthermore, additional external input devices 193 may be communicated with the first vial cavity 158, either directly or via a third configurable flow direction device 195 to allow an operator to introduce a plurality of different substances to the first vial cavity 158, either simultaneously and/or separately. Moreover, a second monitoring device 191 may be provided and configured to be associated with the first Sep-Pak device 138 to monitor a characteristic of the first Sep-Pak device 138, such as the up-take of radioactivity during processing.

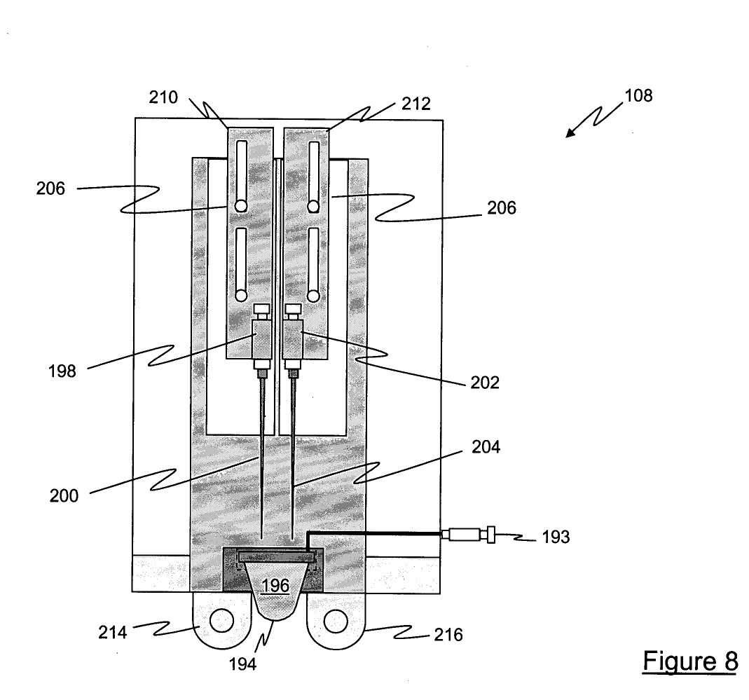

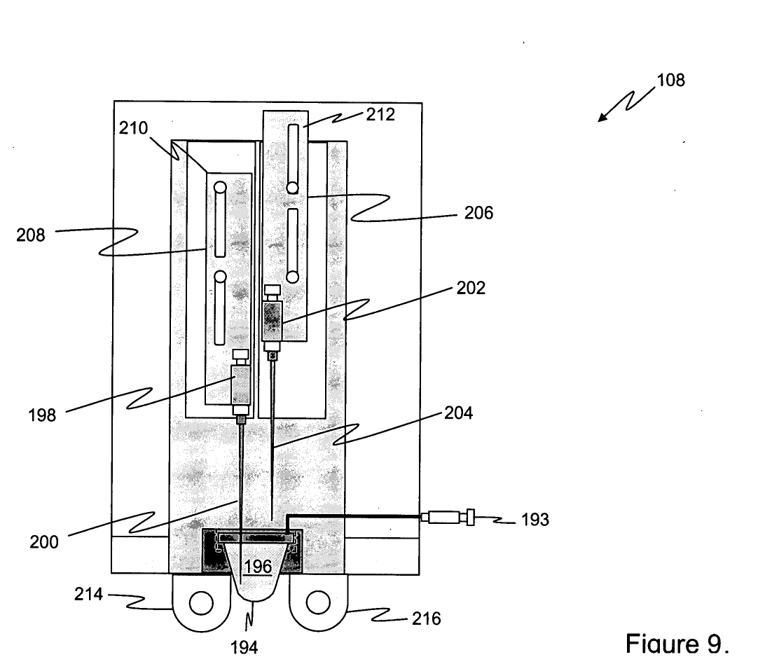

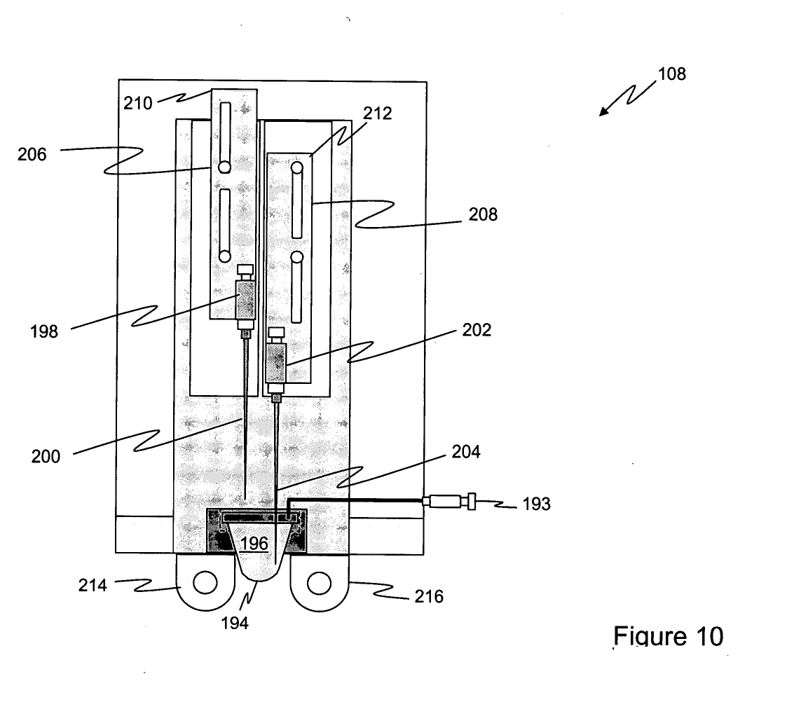

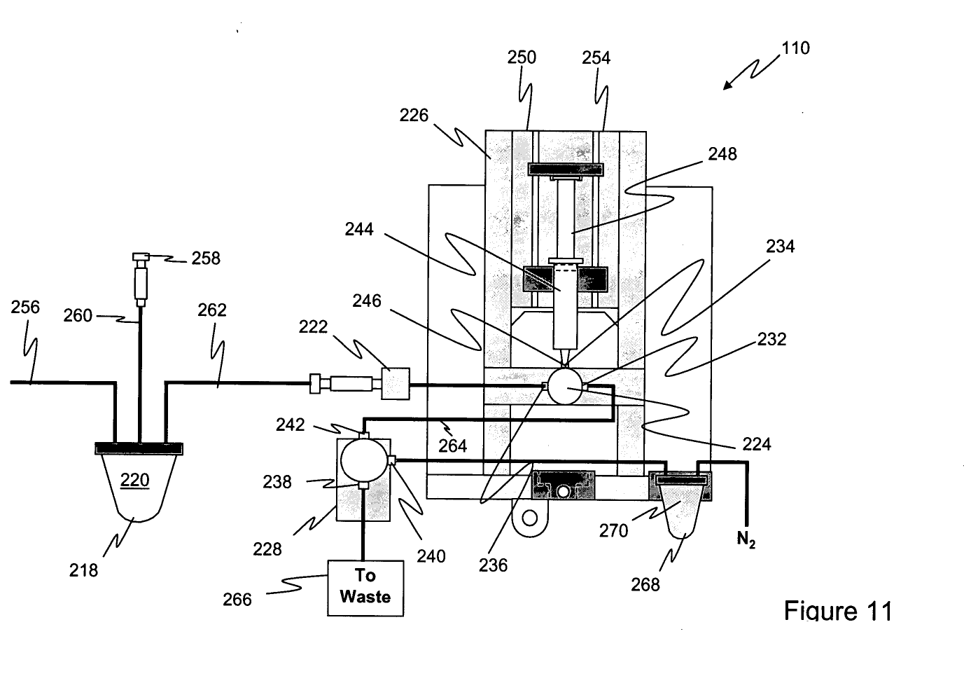

Referring to FIG. 8, FIG. 9 and FIG. 10, the second synthesis station 108 includes a second vial 194 defining a second vial cavity 196, a third needle device 198 having a third needle 200 and a fourth needle device 202 having a fourth needle 204, wherein each of the third needle device 198 and the fourth needle device 202 are separately configurable between a disengaged configuration 206 and an engaged configuration 208 via a third needle actuation device 210 and a fourth needle actuation device 212, respectively. It should be appreciated that when the third needle device 198 is configured into the disengaged configuration 206, the third needle 200 is disposed away from the second vial cavity 196 and when the third needle device 198 is configured into the engaged configuration 208, the third needle 200 is at least partially disposed within the second vial cavity 196. In a similar fashion, when the fourth needle device 202 is configured into the disengaged configuration 206, the fourth needle 204 is disposed away from the second vial cavity 196 and when the fourth needle device 202 is configured into the engaged configuration 208, the fourth needle 204 is at least partially disposed within the second vial cavity 196. The second synthesis station 108 may also include a second temperature sensing device 214 and a second radiation monitoring device 216, wherein the second temperature sensing device 214 may be disposed to sense the temperature of the second vial 194 and the second radiation monitoring device may be disposed to sense any radiation being emitted from the second vial 194. In a similar fashion as the first synthesis station 106, the additional external input devices 193 may also be communicated with the second vial cavity 196, either directly or via the third configurable flow direction device 195 to allow an operator to introduce a plurality of different substances to the second vial cavity 196, either simultaneously and/or separately.

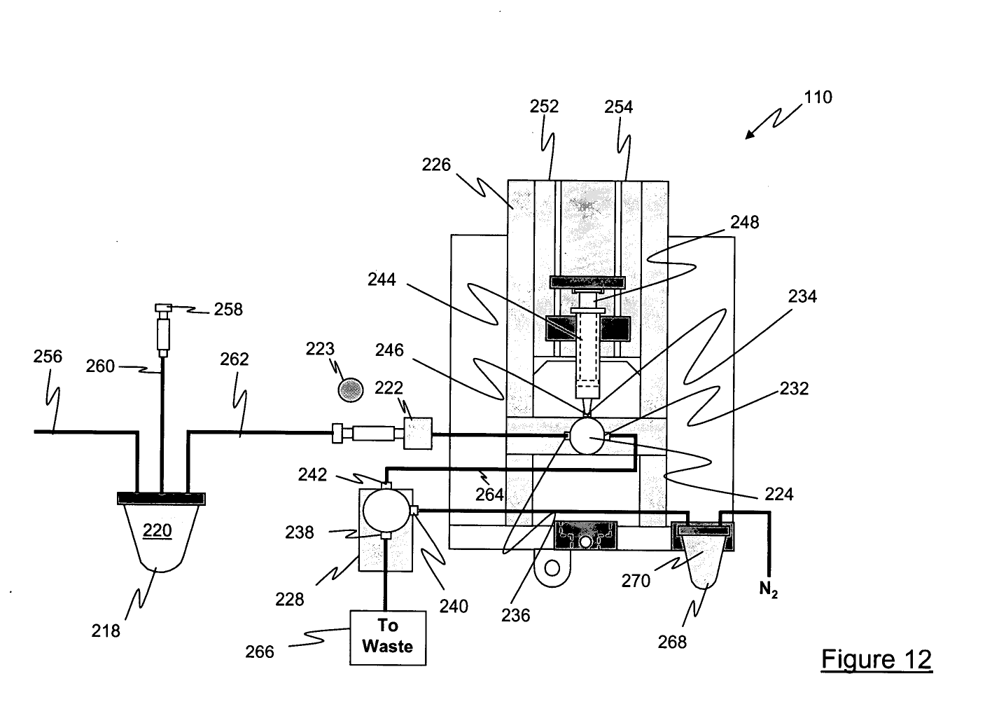

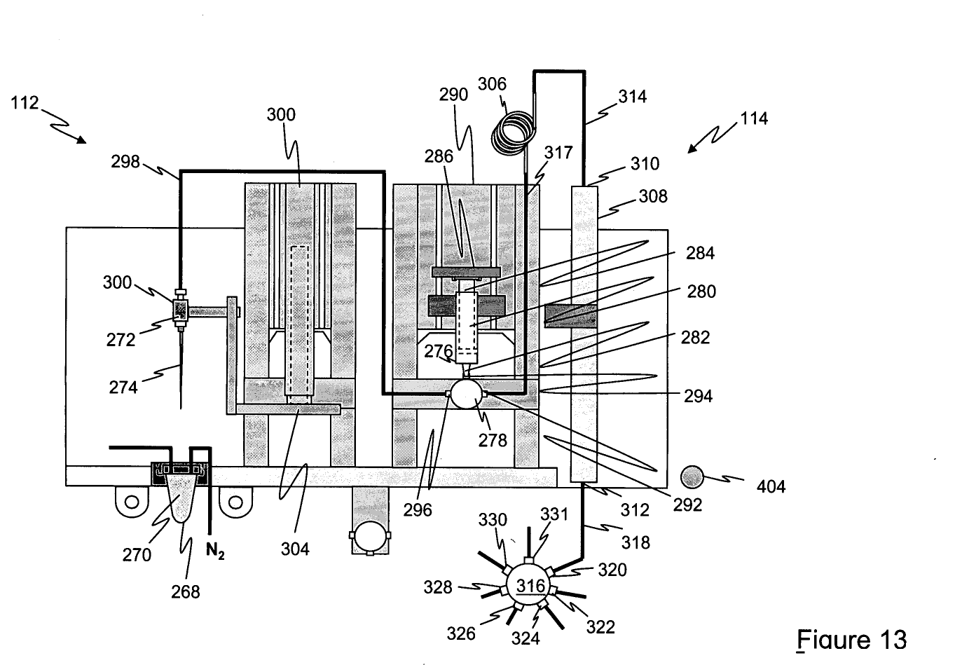

Referring to FIG. 11 and FIG. 12, the third synthesis station 110 includes a third vial 218 defining a third vial cavity 220, a second Sep-Pak device 222, a fourth configurable flow direction device 224, a first syringe device 226 and a fifth configurable flow direction device 228. The fourth configurable flow direction device 224 includes a fourth valve first port 232, a fourth valve second port 234 and a fourth valve third port 236. Similarly, the fifth configurable flow direction device 228 includes a fifth valve first port 238, a fifth valve second port 240 and a fifth valve third port 242. The first syringe device 226 defines a first syringe cavity 244 and includes a first syringe cavity opening 246 and an actuatable plunger portion 248, wherein the actuatable plunger portion 248 is movably disposed within at least a portion of the first syringe cavity 244 and wherein the actuatable plunger portion 248 is controllably configurable between a first plunger configuration 250 and a second plunger configuration 252 via a first plunger actuation device 254. It should be appreciated that the third vial cavity 220 may be communicated with the second needle 166 via a second needle device tube 256. Additionally, the third vial cavity 220 may be communicated with an external input device 258 and the second Sep-Pak device 222 via an external input tube 260 and a second Sep-Pak device tube 262, respectively. The second Sep-Pak device 222 may be further communicated with the fourth valve third port 236, such that a fluid flowing between the third vial 218 and the fourth configurable flow direction device 224 must flow through the second Sep-Pak device 222. Furthermore, the first syringe device 226 may be connected to the fourth configurable flow direction device 224 via the fourth valve second port 234 and the fourth valve first port 232 may be connected to the fifth valve third port 242 of the fifth configurable flow direction device 228 via a fourth valve flow tube 264, wherein the fifth valve first port 238 may be connected to a waste container 266 and the fifth valve second port 240 may be communicated with a fourth vial cavity 270 defined by a fourth vial 268. An additional monitoring device 223 may be provided and configured to be associated with the second Sep-Pak device 222 to monitor a characteristic of the second Sep-Pak device 222, such as the up-take of radioactivity during processing.

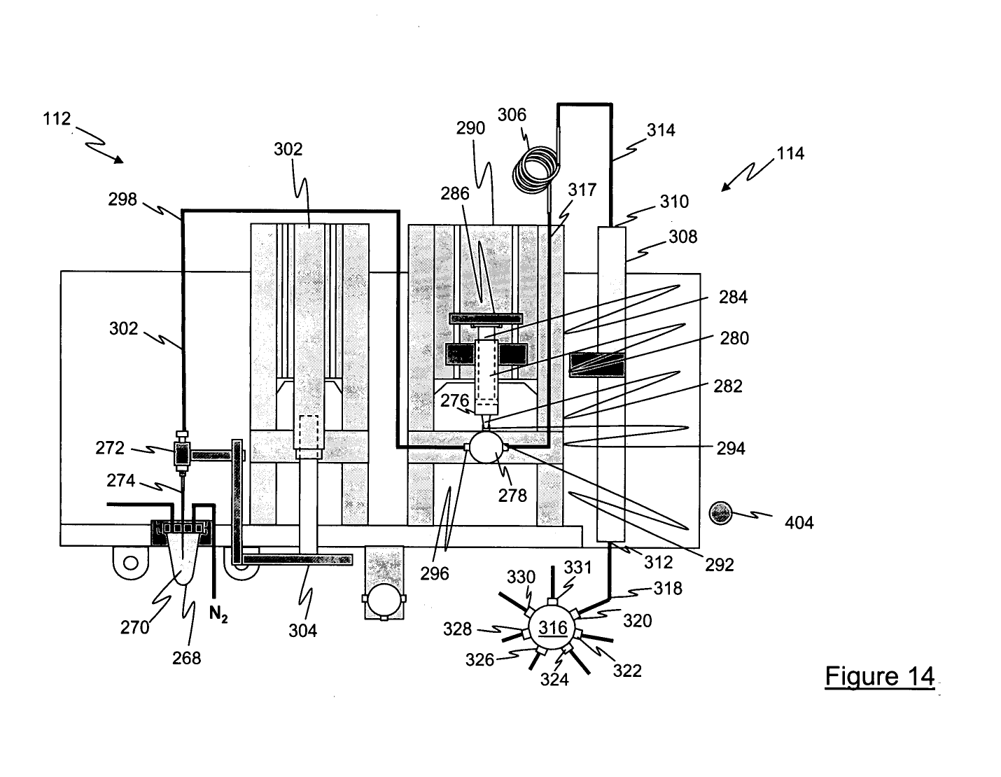

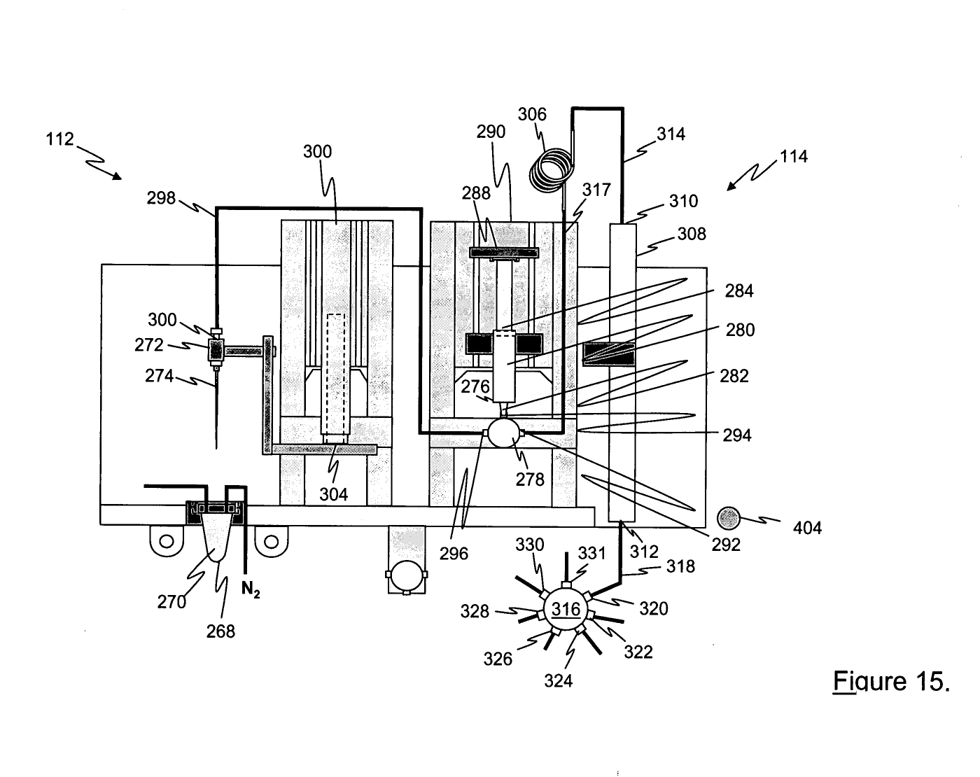

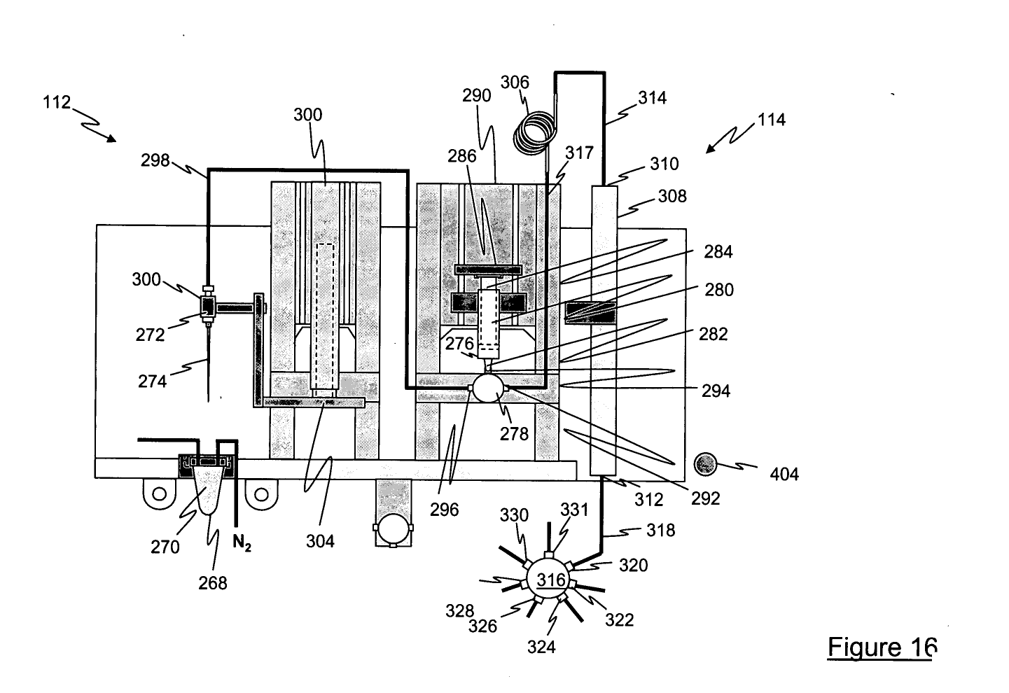

Referring to FIG. 13, FIG. 14, FIG. 15 and FIG. 16, the fourth synthesis station 112 includes the fourth vial 268 which defines the fourth vial cavity 270, a fifth needle device 272 having a fifth needle 274, a second syringe device 276 and a sixth configurable flow direction device 278. The second syringe device 276 defines a second syringe cavity 280 and includes a second syringe cavity opening 282 and an actuatable plunger portion 284, wherein the actuatable plunger portion 284 is movably disposed within at least a portion of the second syringe cavity 280 and wherein the actuatable plunger portion 284 is controllably configurable between a first plunger configuration 286 and a second plunger configuration 288 via a second plunger actuation device 290. The sixth configurable flow direction device 278 includes a sixth valve first port 292, a sixth valve second port 294 and a sixth valve third port 296, wherein the sixth valve third port 296 may be communicated with the fifth needle 274 via a fifth needle flow tube 298 and the sixth valve second port 294 may be communicated with the second syringe device 276 via the second syringe cavity opening 282. It should be appreciated that the fifth needle device 272 may also be controllably configurable between a disengaged configuration 300 and an engaged configuration 302 via a fifth needle actuation device 304 such that when the fifth needle device 272 is configured into the engaged configuration 302, at least a portion of the fifth needle 274 is disposed within the fourth vial cavity 270 and when the fifth needle device 272 is configured into the disengaged configuration 300, the fifth needle 274 is disposed away from the fourth vial cavity 270.

The HPLC station 114 is also shown and includes an HPLC loop 306 connected to an HPLC column 308, wherein the HPLC column 308 includes an HPLC inlet port 310 and an HPLC outlet port 312. The HPLC loop 306 may be connected between the HPLC inlet port 310 and the sixth valve first port 292 of the sixth configurable flow direction device 278 via an HPLC inlet tube 314 and an HPLC loop tube 317, respectively. The HPLC outlet port 312 may be further connected with a seventh configurable flow direction device 316 via an HPLC outlet tube 318, wherein the seventh configurable flow direction device 316 is shown as a seven way directional flow valve having a seventh valve first port 320, a seventh valve second port 322, a seventh valve third port 324, a seventh valve fourth port 326, a seventh valve fifth port 328, a seventh valve sixth port 330 and a seventh valve seventh port 331. A fourth monitoring device 404, such as a radioactivity detector and/or an Ultra Violet (UV) detector may be included and disposed to monitor the input and/or output from the HPLC column 308. It should be appreciated that the mobile phase for the HPLC station 114 may be introduced onto the HPLC column 308 by passing through the HPLC loop 306 via two flow pathways, one when the material to be purified may be loaded onto the HPLC loop 306, wherein the HPLC loop 306 is not connected to the mobile phase and column and the other when the material to be purified is introduced onto the HPLC column 308 by having the mobile phase flow through the HPLC loop 306 as shown herein.

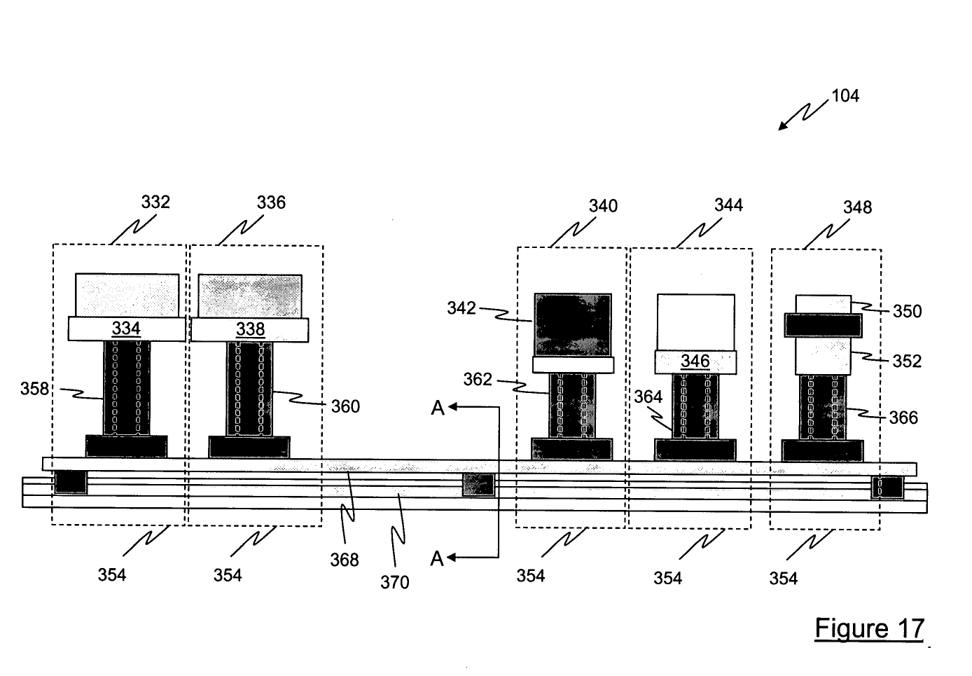

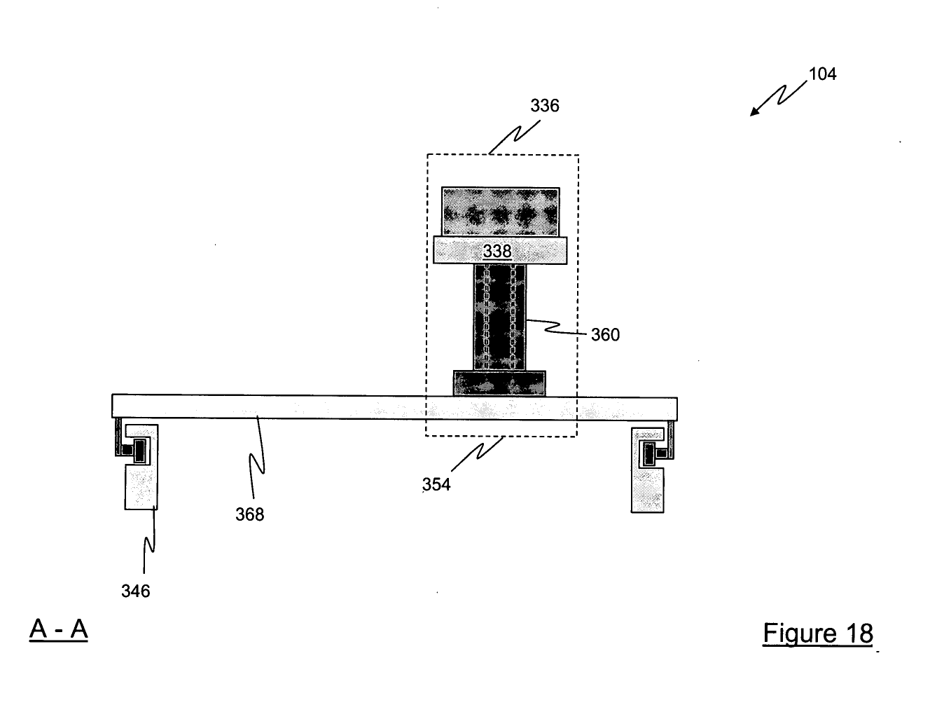

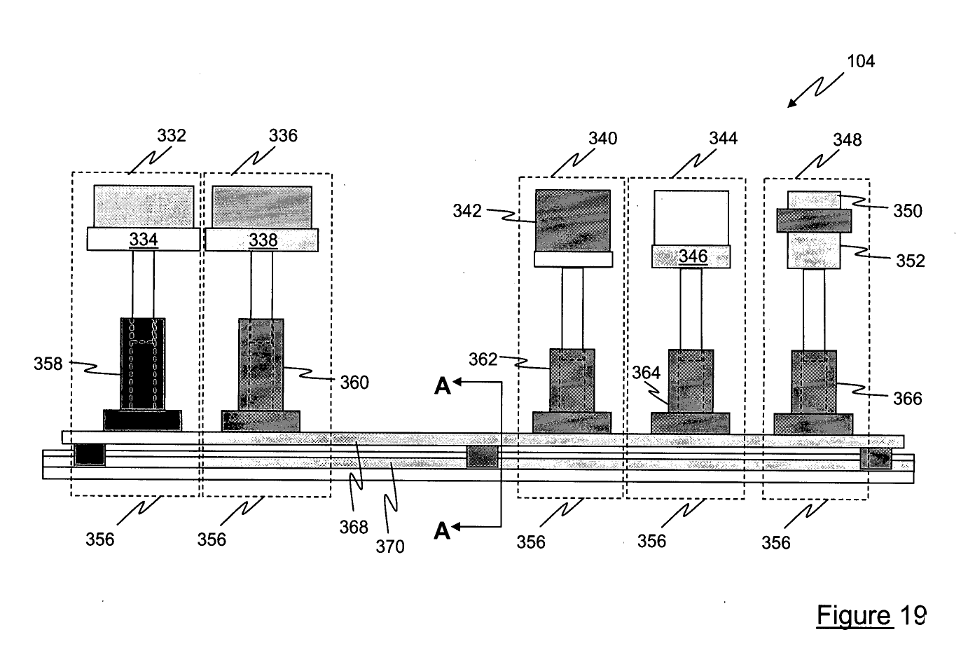

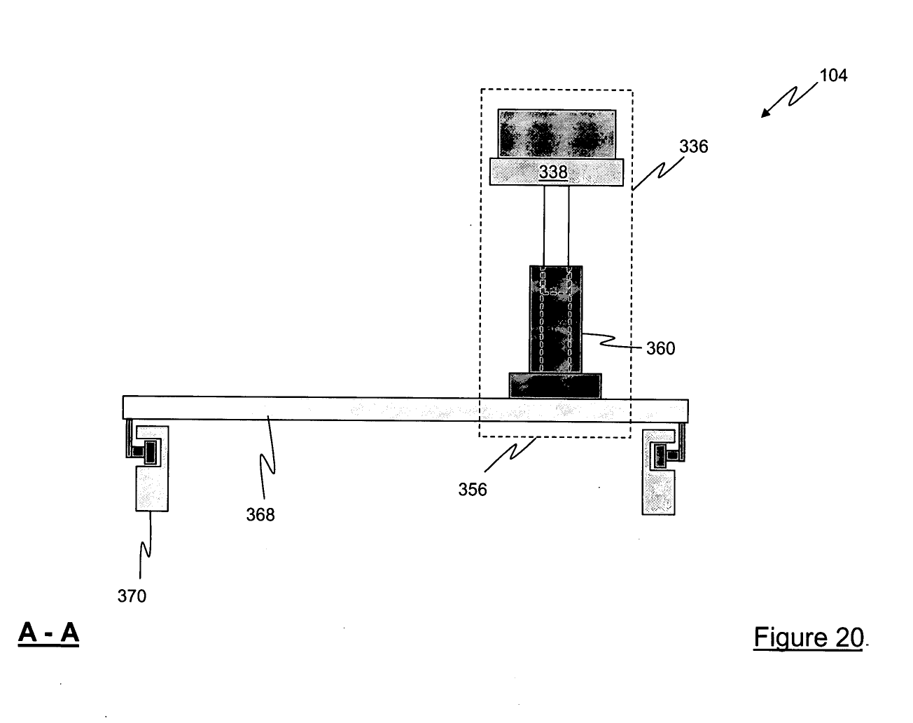

Referring to FIG. 17, FIG. 18, FIG. 19 and FIG. 20, the second synthesis portion 104 includes a first bathing station 332 having a first bathing structure 334, a second bathing station 336 having a second bathing structure 338, a third bathing station 340 having a third bathing structure 342, a shielding station 344 having a shielding structure 346 and a microwave station 348 having a microwave device 350 supported by a microwave structure 352, wherein each of the first bathing structure 334, the second bathing structure 338, the third bathing structure 342, the shielding structure 346 and the microwave structure 352 may be configurable between a contracted configuration 354 and an extended configuration 356 via a first bathing structure actuation device 358, a second bathing structure actuation device 360, a third bathing structure actuation device 362, a shielding structure actuation device 364 and a microwave structure actuation device 366. The second synthesis portion 104 also includes a movably configurable support platform 368 which is movable in at least two (2) dimensions (XY plane) and may be movable in three (3) dimensions (XYZ plane) via a support platform actuation device 370. Each of the first bathing structure 334, the second bathing structure 338, the third bathing structure 342, the shielding structure 346 and the microwave structure 352 may be non-movably connected to the support platform 368 to allow each of the first bathing station 332, the second bathing station 336, the third bathing station 340, the shielding station 344 and the microwave station 348 to be separately and controllably associated and disassociated with each of the elements of the first synthesis station 106, the second synthesis station 108, the third synthesis station 110 and the fourth synthesis station 112.

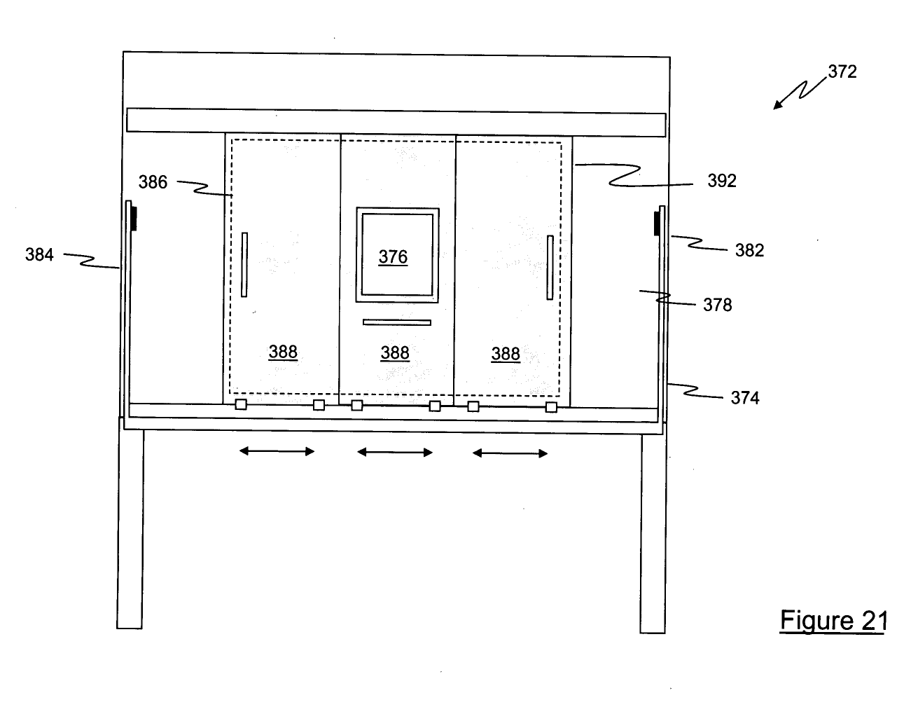

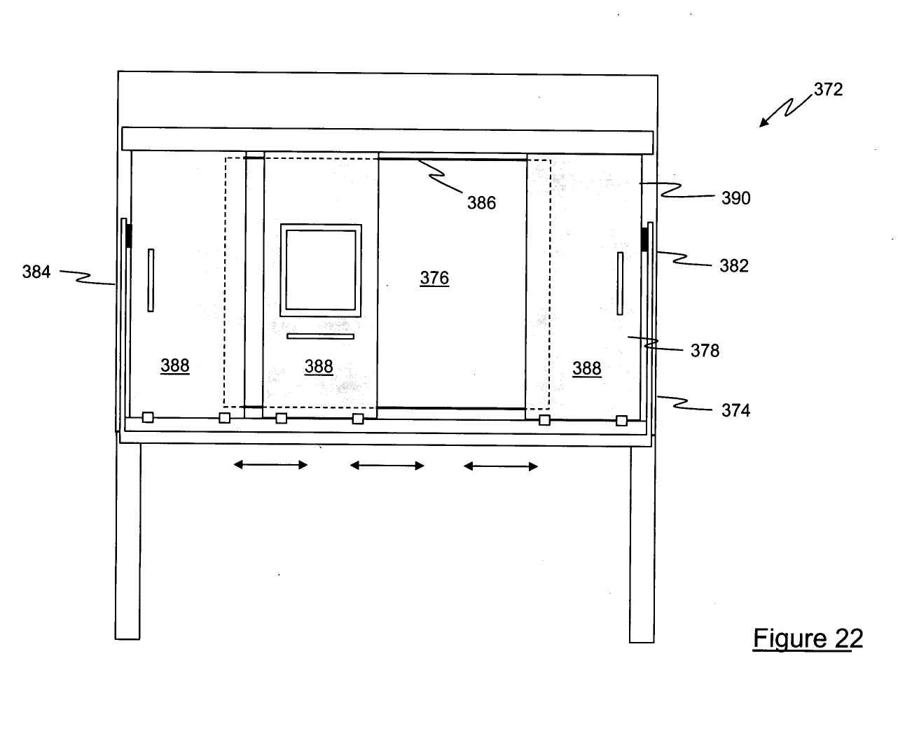

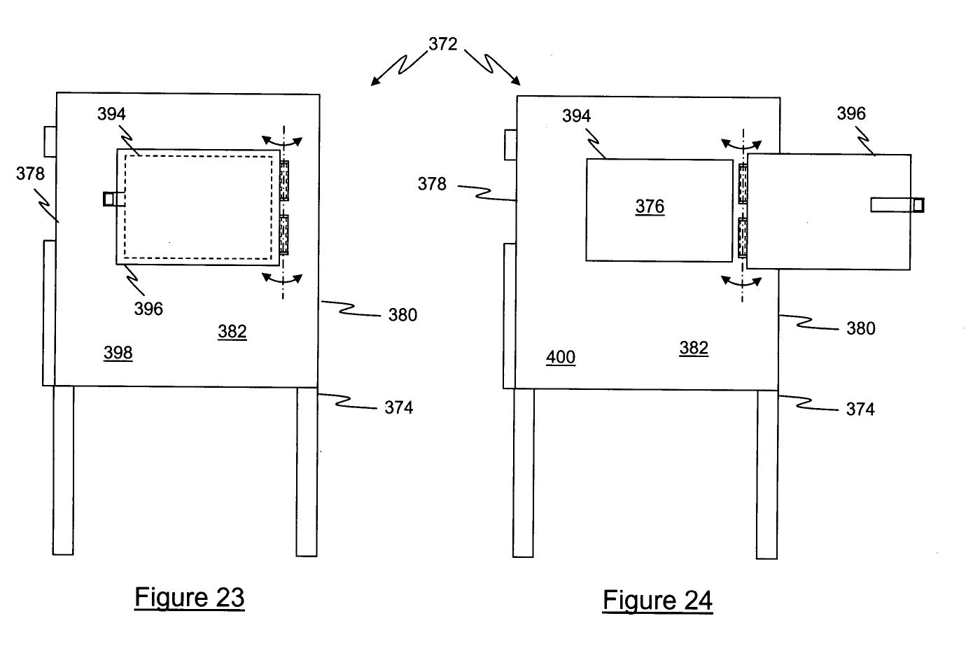

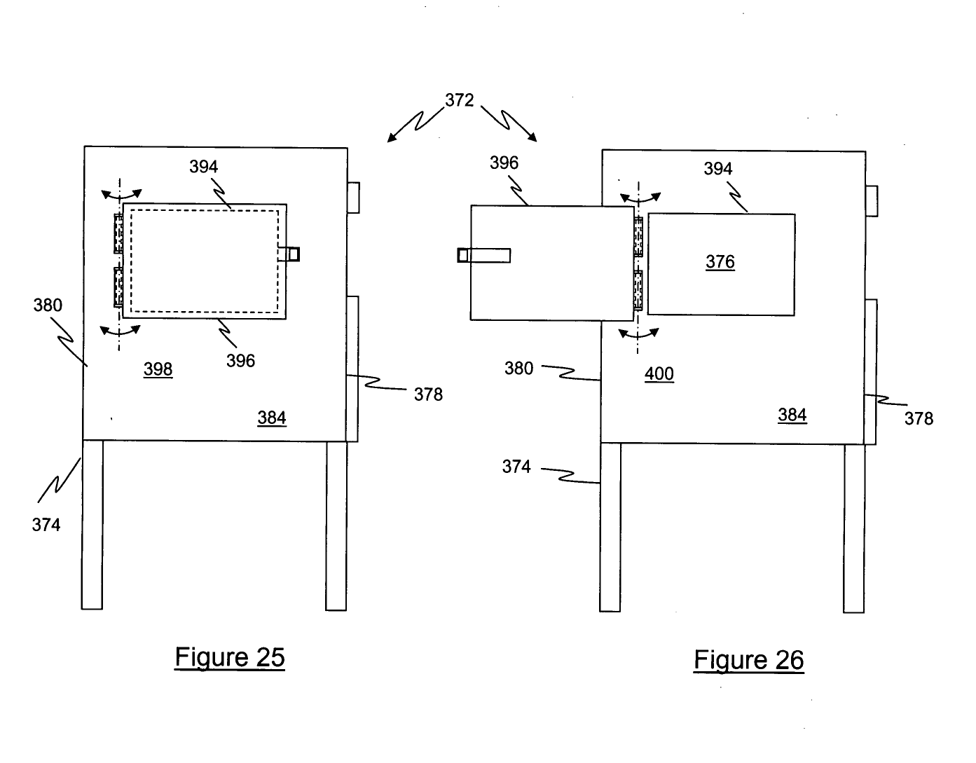

Additionally, referring to FIGS. 21-26, the remote controlled synthesis device (RCSD) 100 also includes a shielding enclosure 372 having an enclosure structure 374, wherein the enclosure structure 374 defines an enclosure cavity 376 and includes an enclosure front portion 378, an enclosure rear portion 380, an enclosure right side 382 and an enclosure left side 384. The enclosure front portion 378 defines an enclosure front opening 386 having a plurality of front opening doors 388 each of which are configurable between an open configuration 390 and a closed configuration 392 to allow access to the enclosure cavity 376 via the enclosure front opening 386. Furthermore, the enclosure right side 382 and the enclosure left side 384 each define an enclosure side opening 394 having an enclosure side door 396 which is configurable between a closed configuration 398 and an open configuration 400 to allow access to the enclosure cavity 376 via either the enclosure right side 382 and/or the enclosure left side 384.

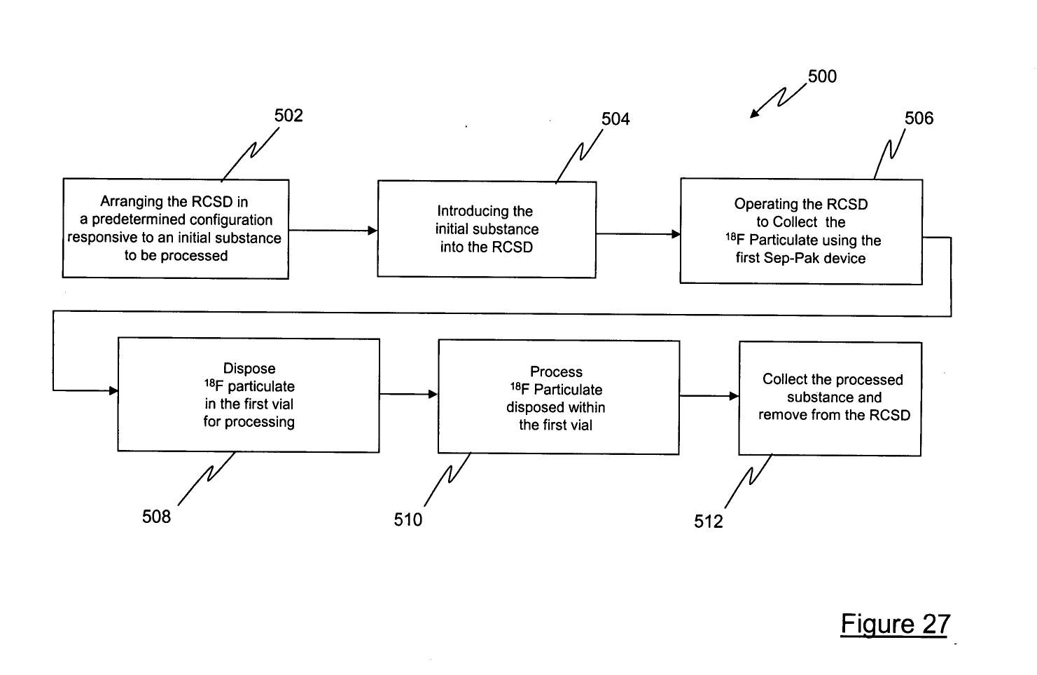

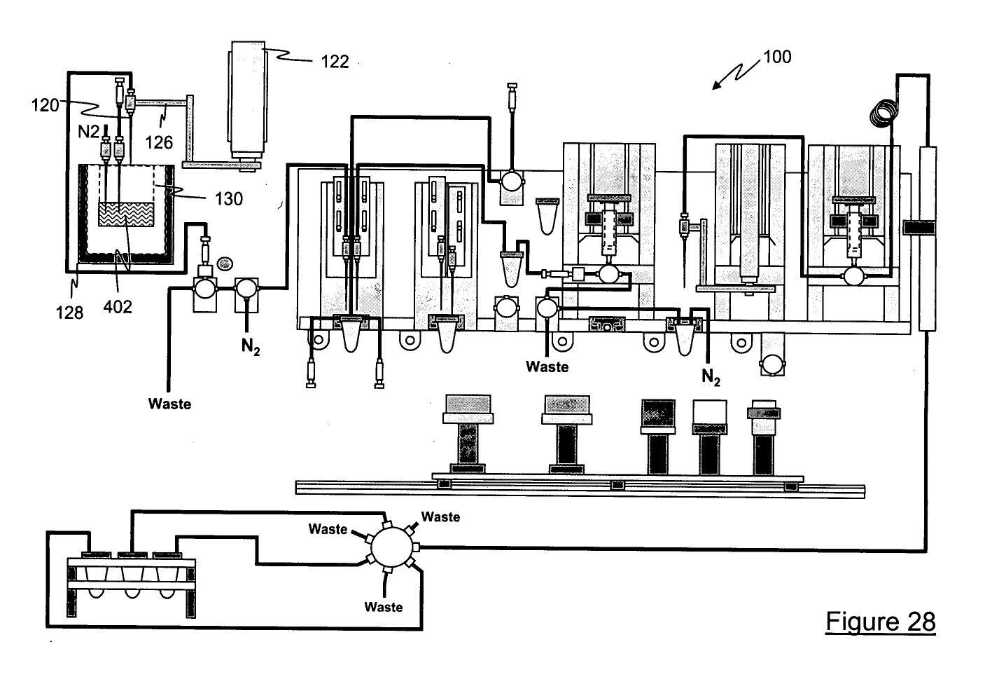

Referring to FIG. 27, a block diagram illustrating a method 500 for implementing the remote-controlled synthesis device 100 is shown in terms of a typical ¹⁸F synthesis and purification process and includes obtaining and physically configuring the remote-controlled synthesis device 100 for the ¹⁸F synthesis and purification process as shown in operational block 502. This may be accomplished by assembling the glassware and solid phase cartridges as needed, connecting the needles as appropriate, activating the N₂ and pneumatic device(s), the controller device(s), any required detector(s) and any other equipment as required to perform the process, such as the positioning device(s), radiation detector(s) and UV detector(s). Referring to FIG. 28, the lines of the RCSD 100 may be rinsed with CH₃CN and dried with N₂ to prevent product contamination and oxidation and a shipping vial containing an ¹⁸F compound 402 may be introduced into the RCSD 100 by removing the ¹⁸F compound 402 from a shielded shipping container and disposing the ¹⁸F compound 402 within the container holding device cavity 130 of the container holding device 128, as shown in operational block 504. This may be accomplished manually via laboratory personnel or this may be accomplished via an automated process which would allow for the ¹⁸F compound 402 to be introduced directly into the RCSD 100 from a cyclotron device and/or a shielded shipping container without any direct contact by laboratory personnel.

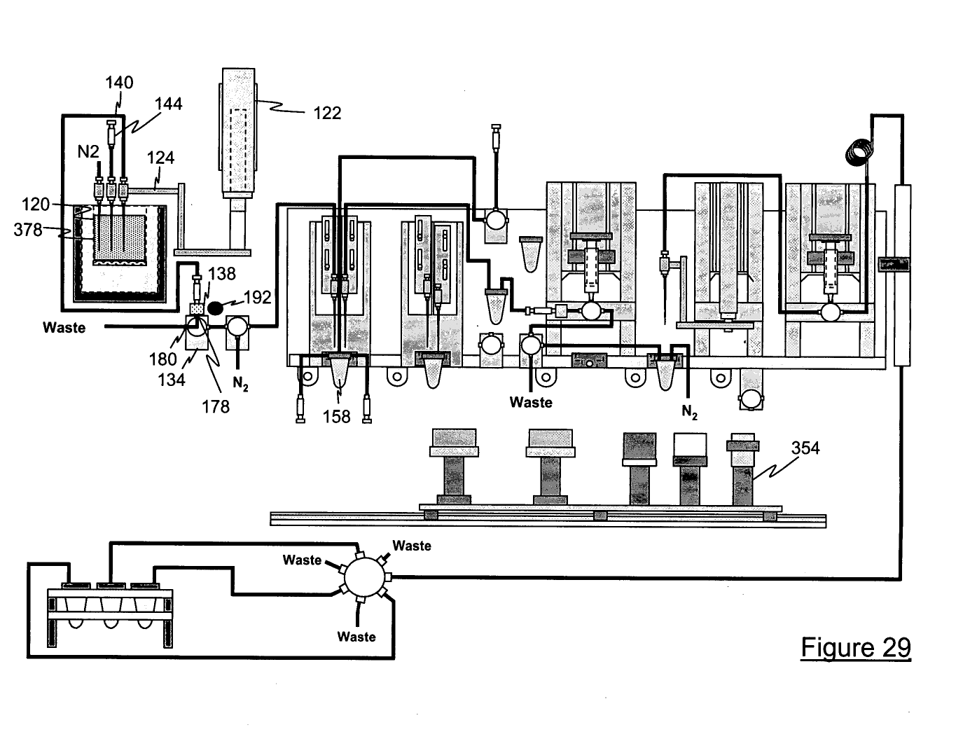

Referring to FIG. 29, the input needle actuation device 122 is configured from the disengaged configuration 126 into the engaged configuration 124 such that the input needle 120 is at least partially disposed within the shipping vial 378 to be in contact with the ¹⁸F compound 402 contained therein. The ¹⁸F particulate contained within the ¹⁸F compound is then collected, as shown in operational block 506. This may be accomplished by configuring the first configurable flow direction device 134 such that the first valve second port 178 is communicated with the first valve third port 180. The N₂ pressure within the shipping vial 378 is increased to cause the ¹⁸F compound contained within the shipping vial 378 to flow through the first needle 120, out of the shipping vial 378 into the first flow tube 140, through the first Sep-Pak device 138, into the first valve second port 178, out of the first valve third port 180 and into a waste container. The flow of the ¹⁸F compound through the first Sep-Pak device 138 may be controlled via the N₂ pressure such that the ¹⁸F contained within the solution is captured and retained by the first Sep-Pak device 138 while the effluent is directed to and deposited within the waste container via the first configurable flow direction device 134. During this process, the uptake of radioactivity of the ¹⁸F contained within the first Sep-Pak device 138 may be monitored using the second radiation monitoring device 191 disposed to be associated with the first Sep-Pak device 138.

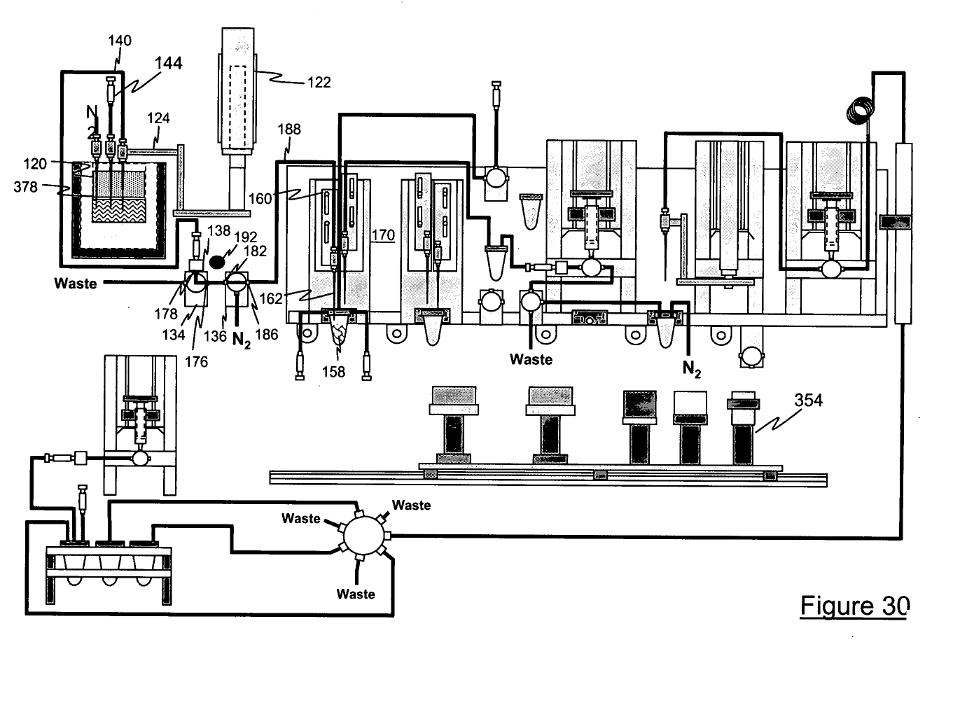

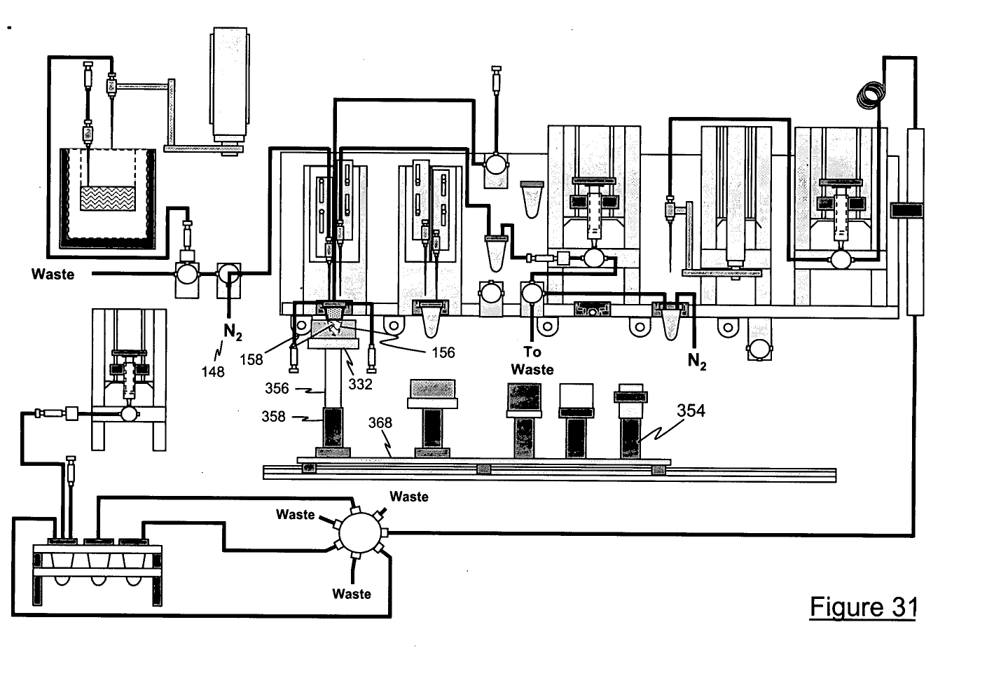

The ¹⁸F collected and contained within the first Sep-Pak device 138 may then be disposed within the first vial cavity 158 for processing, as shown in operational block 508. Referring to FIG. 30, this may be accomplished by configuring the first configurable flow direction device 134 such that the first valve second port 178 is communicated with the first valve first port 176, configuring the second configurable flow direction device 136 such that the second valve first port 182 is communicated with the second valve third port 186 and adding a K₂CO₃ solution and a K222 solution (kryptofix 222) to the shipping vial 378 via the first external input device 144. The first needle device 160 is configured between the disengaged configuration 168 and the engaged configuration 170 such that the first needle 162 is at least partially disposed within the first vial cavity 158. As above, the N₂ pressure within the shipping vial 378 is increased to cause the combined K₂CO₃ solution and K222 solution to be directed out of the shipping vial 378 into the input needle 120 through the first flow tube 140, through the first Sep-Pak device 138, through the first configurable flow direction device 134, through the second configurable flow direction device 136, into the third flow tube 188, out of the first needle 162 and into the first vial cavity 158. The flow of the combined K₂CO₃ solution and K222 solution through the first Sep-Pak device 138 may be controlled via the N₂ pressure to cause the ¹⁸F collected and retained within the first Sep-Pak device 138 to become dislodged from the first Sep-Pak device 138 and to flow into the first vial cavity 158. It should be appreciated that the K₂CO₃ solution and the K222 solution may be added to the shipping vial 378 via the first external input device 144 and the process repeated if desired.

Similarly, a CH₃CN solution may then be added to the shipping vial 378 via the first external fluid input device 144 and the N₂ pressure is increased to cause the CH₃CN solution to be directed out of the shipping vial 378, into the input needle 120, through the first flow tube 140, through the first Sep-Pak device 138, through the first configurable flow direction device 134, through the second configurable flow direction device 136, into the third flow tube 188, out of the first needle 162 and into the first vial cavity 158 along with the K₂CO₃ solution, the K222 solution and the ¹⁸F. The CH₃CN solution, the K₂CO₃ solution, the K222 solution and the ¹⁸F disposed within the first vial cavity 156 may then be processed, as shown in operational block 510. Referring to FIG. 31, this may be accomplished by raising the pressure within the first vial cavity 158 using the N₂ supply device 148 and configuring the support platform 368 such that the first bathing station 332 is disposed directly below the first vial 156 and the first bathing structure 332 is raised such that the first vial 156 is immersed within a hot oil bath disposed within the first bathing structure 332. This may be accomplished by operating the first bathing structure actuation device 358 to cause the first bathing structure 332 to be configured from the contracted configuration 354 into the extended configuration 356. The first bathing structure 332 may also include an element which maintains a desired temperature of the fluid contained with the first bathing structure 332. For example, in this embodiment the oil contained within the first bathing structure 332 may be maintained at approximately 90° C. and may be monitored via the first temperature sensing device 190.

Using the heat, vacuum and the N₂, the CH₃CN—H20 solution is evaporated (azeotroped) until the amount of solution remaining within the first vial cavity 158 is approximately 0.3 ml. Once the amount of solution remaining within the first vial cavity 158 is approximately equal to 0.3 ml, the N₂ pressure within the first vial 156 is decreased and a 0.5 ml solution of CH₃CN is added to the solution remaining within the first vial cavity 158. This may be accomplished by adding the 0.5 ml solution of CH₃CN to the first vial cavity 158 via an external input device 193. The pressure within the first vial cavity 158 is again increased and, using the heat and the N₂ pressure, the solution contained within the first vial cavity 158 is evaporated (azeotroped) until the amount of solution remaining within the first vial cavity 158 is approximately equal to 0.1 ml. A dry vacuum device may then be used to assist in the azeotropic removal of any solvents from within the first vial cavity 158. The N₂ pressure within the first vial cavity 158 is again decreased and a 0.5 ml solution of CH₃CN is again added to the solution remaining within the first vial cavity 158. The pressure within the first vial cavity 158 is again increased and, using the heat and the N₂ pressure, the solution contained within the first vial cavity 158 is evaporated (azeotroped) until the amount of solution remaining within the first vial cavity 158 is approximately equal to 0.1 ml. This process may be repeated an additional time if desired.

The first vial 156 may be removed from the oil bath by operating the first bathing structure actuation device 358 to lower the first bathing structure 334 from the extended configuration 356 into the contracted configuration 354. The N₂ pressure within the first vial cavity 158 is decreased and the vacuum supply device is activated to create a full vacuum within the first vial cavity 158 for a predetermined period of time dependent upon the process, in this case approximately six (6) minutes. The full vacuum within the first vial cavity 158 is removed and the residue of K₂CO₃ and K222 containing the ¹⁸F remaining within the first vial cavity 158 is dissolved within a solution of CH₃CN containing a substrate or precursor for radiolabeling (to react with the ¹⁸F) which may be added to the first vial cavity 158 via an external input device. The first bathing structure actuation device 358 is again operated to raise the first bathing structure 334 from the contracted configuration 354 into the extended configuration 356 to immerse the first vial 156 into the hot oil contained within the first bathing structure 334. The first vial 156 is disposed in the hot oil for a predetermined amount of time again dependent upon the process, in this case approximately eight (8) minutes, after which the first bathing structure actuation device 358 is again operated to lower the first bathing structure 334 from the extended configuration 356 into the contracted configuration 354.

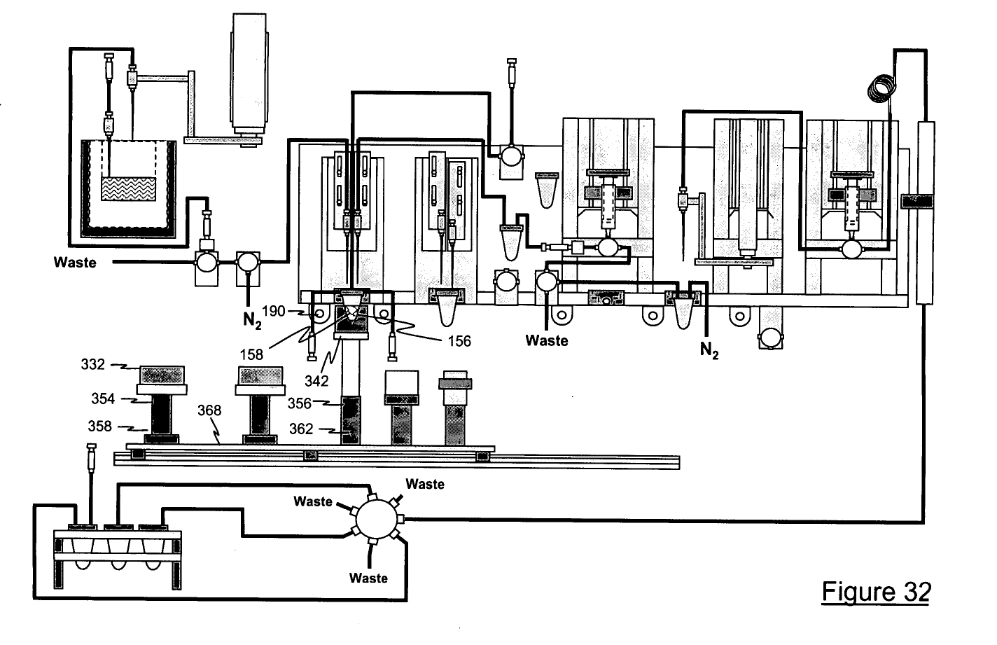

Referring to FIG. 32, the support platform 368 is configured such that the third bathing structure 342 is disposed directly below the first vial 156 and the third bathing structure actuation device 362 is operated to raise the third bathing structure 342 from the contracted configuration 354 into the extended configuration 356 such that the first vial 156 is immersed in a cooling substance disposed within the third bathing structure 342. The outside surface temperature of the first vial 156 may be monitored via the first temperature sensing device 190 and when the outside surface temperature of the first vial 156 is less than 50° C., the first vial 156 is removed from the cooling bath by lowering the third bathing structure 342 from the extended configuration 356 into the contracted configuration 354. The remaining solution contained within the first vial cavity 158 may then be diluted by adding 3 ml of H₂O to the first vial cavity 158. The introduction of additional fluids into the first vial cavity 158 may be accomplished via an external input device 193.

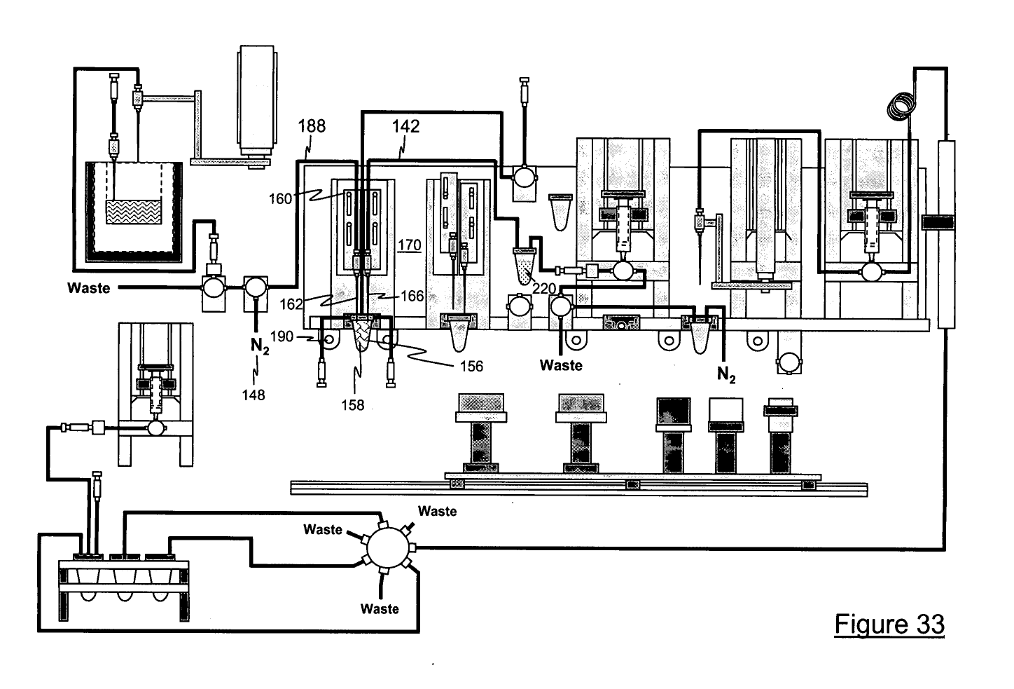

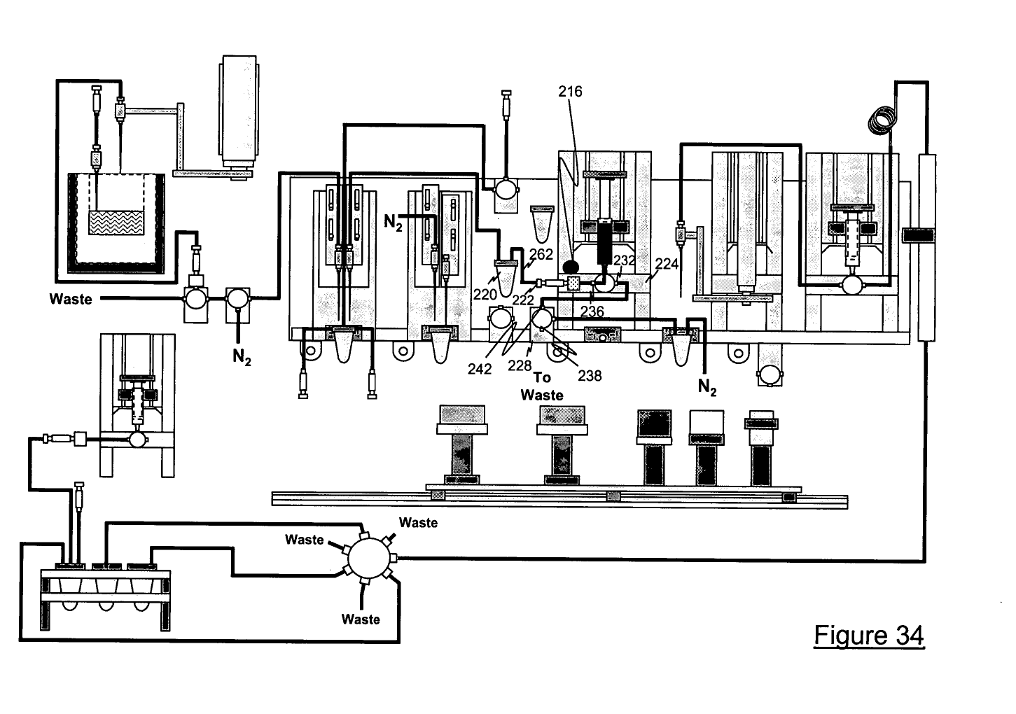

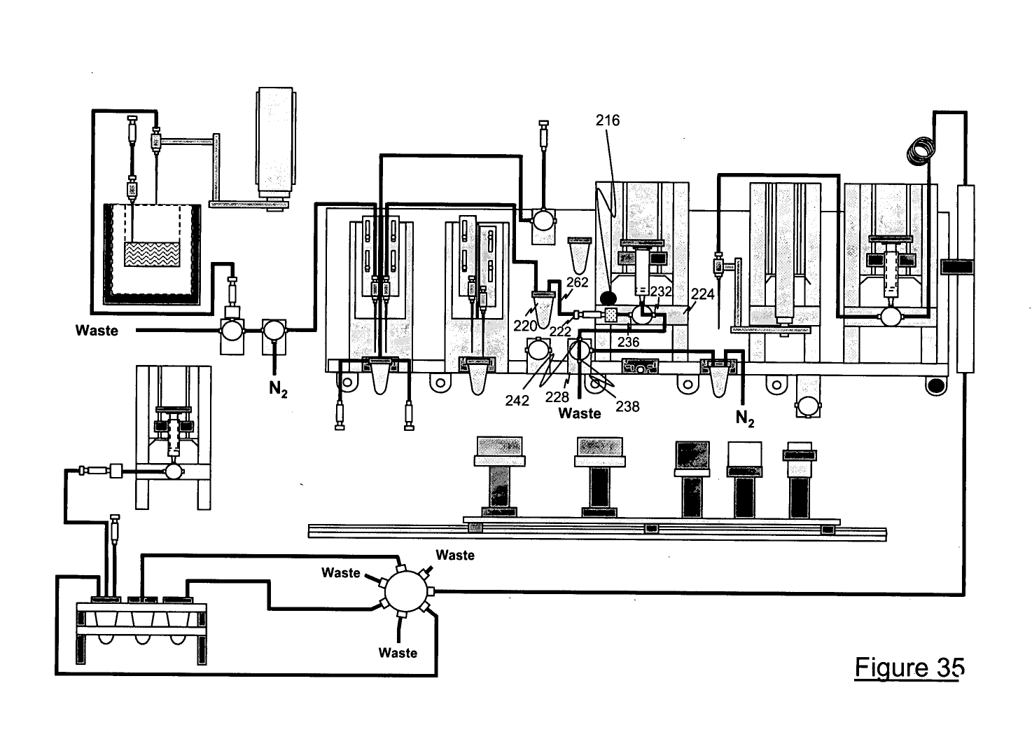

Referring to FIG. 33, the second needle device 164 may then be configured from the disengaged configuration 168 into the engaged configuration 170 such that the second needle 166 is disposed within the first vial cavity 158 and the N₂ pressure within the first vial cavity is increased to cause the diluted solution contained within the first vial cavity 158 to be directed into the second needle 166 and to flow through the second needle flow tube 142 and into the third vial cavity 220. An additional 3 ml of H₂O may be disposed within the first vial cavity 158 and directed to flow into the second needle 166, through the second needle flow tube 142 and into the third vial cavity 220 with the diluted solution. Referring to FIG. 34 and FIG. 35, at this point approximately 10 ml of H₂O is added to the third vial cavity 220 via the external input device 258, the fourth configurable flow direction device 224 is configured such that the fourth valve third port 236 is communicated with the fourth valve second port 234 and the fifth configurable flow direction device 228 is configured such that the fifth valve first port 238 is communicated with the fifth valve third port 242.

The first syringe device 226 is then operated to cause the ¹⁸F compound contained within the third vial cavity 220 to flow into the second Sep-Pak device tube 262 and through the second Sep-Pak device 222 such that the ¹⁸F is retained with the second Sep-Pak device 222 and such that the remaining effluent is contained within the first syringe cavity 244. The fourth configurable flow direction device 224 is then configured such that the fourth valve third port 236 is communicated with the fourth valve first port 232 and the first syringe device 226 is operated such that the effluent is directed out of the fourth valve first port 232 and into the fifth valve third port 242 where the effluent is directed out of the fifth valve first port 238 to waste. Similarly as discussed hereinabove, during this process the uptake of radioactivity of the ¹⁸F in the second Sep-Pak device 222 may be monitored using a second radiation monitoring device 216. In order to ensure that all of the ¹⁸F is removed from the third vial cavity 220 and collected by the second Sep-Pak device 222, an additional 10 ml of H₂O may be added to the third vial cavity 220 via the external input device 258 and the first syringe device 226 may again be operated to cause any remaining ¹⁸F compound contained within the third vial cavity 220 to flow into the second Sep-Pak device tube 262 and through the second Sep-Pak device 222 such that any remaining ¹⁸F is retained within the second Sep-Pak device 222 and such that the remaining effluent is directed to waste.

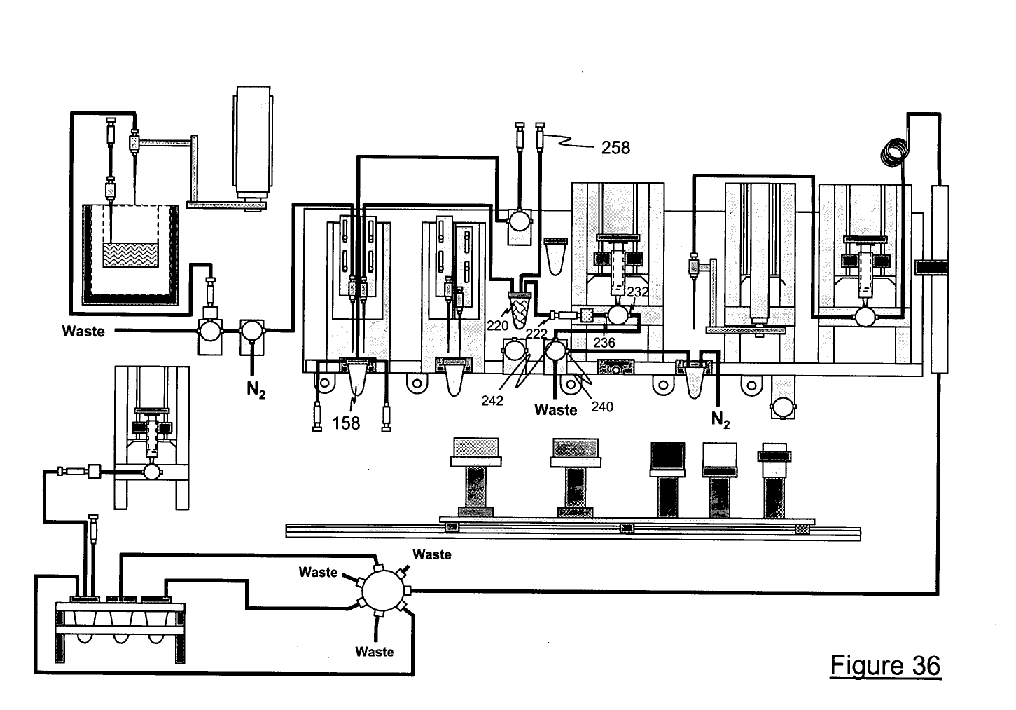

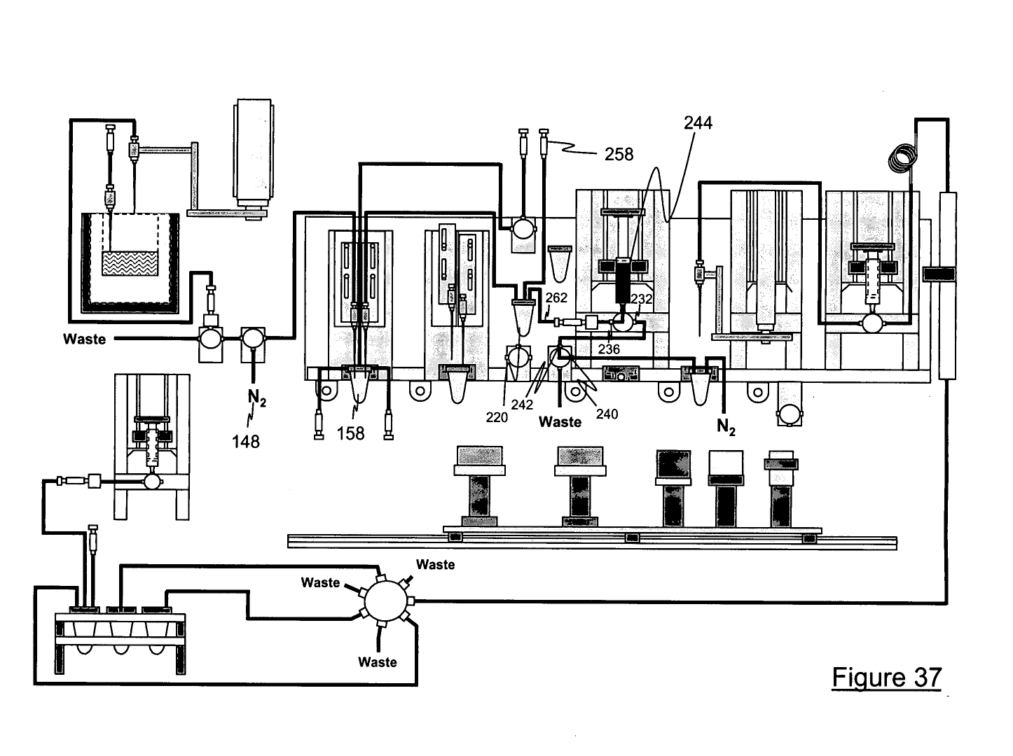

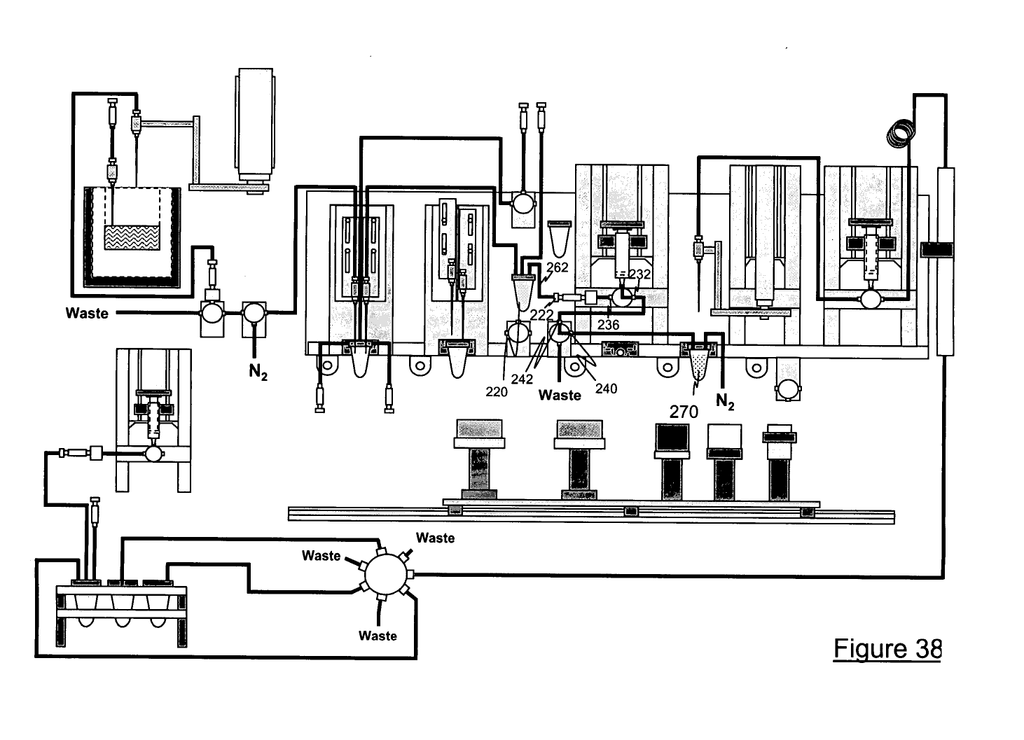

Referring to FIG. 36, at this point approximately 3 ml of CH₃CN solvent is added to the third vial cavity 220 via the external input device 258 and the fourth configurable flow direction device 224 is configured such that the fourth valve third port 236 is communicated with the fourth valve second port 234. Referring to FIG. 37, the first syringe device 226 is operated to cause the CH₃CN to flow out of the third vial cavity 220, through the second Sep-Pak device 222 via the second Sep-Pak device tube 262 and into the first syringe cavity 244, such that any 18F collected and retained by the second Sep-Pak device 222 is dislodged and flows out of the second Sep-Pak device 222 with the CH₃CN. Referring to FIG. 38, the fourth configurable flow direction device 224 is then configured such that the fourth valve second port 234 is communicated with the fourth valve first port 232 and the first syringe device 226 is operated to cause the CH₃CN to flow out of the first syringe cavity 244, into the fourth valve second port 234, out of the fourth valve first port 232, into the fifth valve third port 242, out of the fifth valve second port 240 and into the fourth vial cavity 270. This process may be repeated to ensure that any 18F particulate residue retained within the second Sep-Pak device 222 is dislodged and removed.

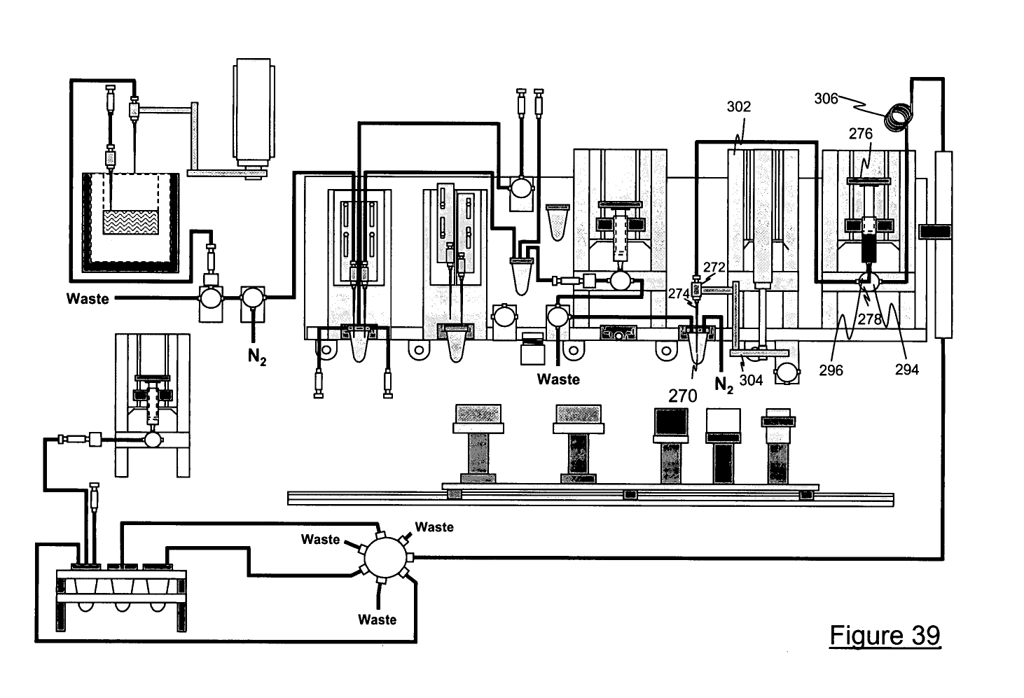

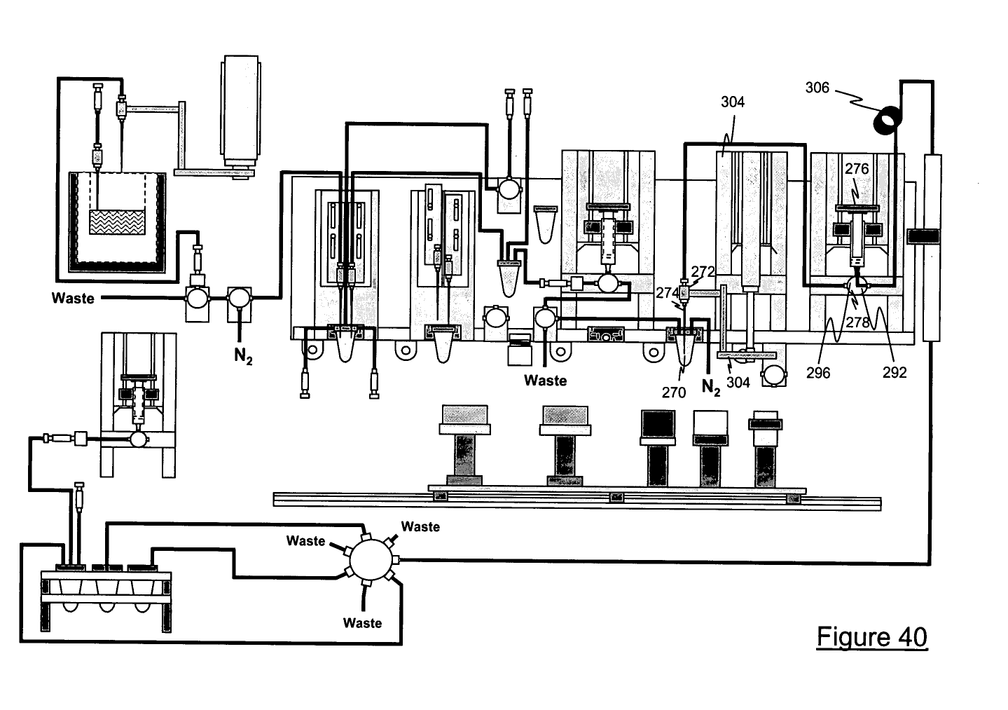

Referring to FIG. 39, the fifth needle actuation device 304 is operated to controllably configure the fifth needle device 272 between the disengaged configuration 300 and the engaged configuration 302 such that the fifth needle 274 is disposed within the fourth vial cavity 270 to be in contact with the CH₃CN solvent and the ¹⁸F contained therein. The sixth configurable flow direction device 278 is configured such that the sixth valve second port 294 is communicated with the sixth valve third port 296 and the second syringe device 276 is operated to cause the actuatable plunger portion 284 to traverse the second syringe cavity 280 such that the CH₃CN solvent and the ¹⁸F contained within the fourth vial cavity 270 is drawn into the second syringe cavity 280. Referring to FIG. 40, the sixth configurable flow direction device 278 is the reconfigured such that the sixth valve second port 294 is communicated with the sixth valve first port 292 and the second syringe device 276 is operated to introduce the CH₃CN solvent and the ¹⁸F contained within the second syringe cavity 280 into the HPLC loop 306.

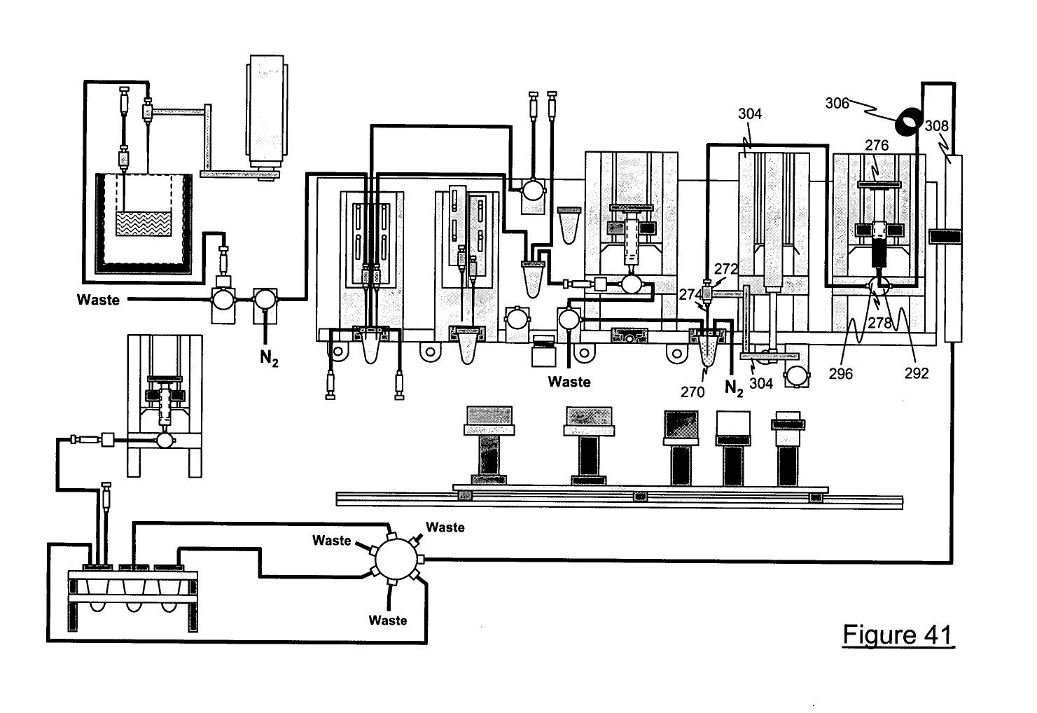

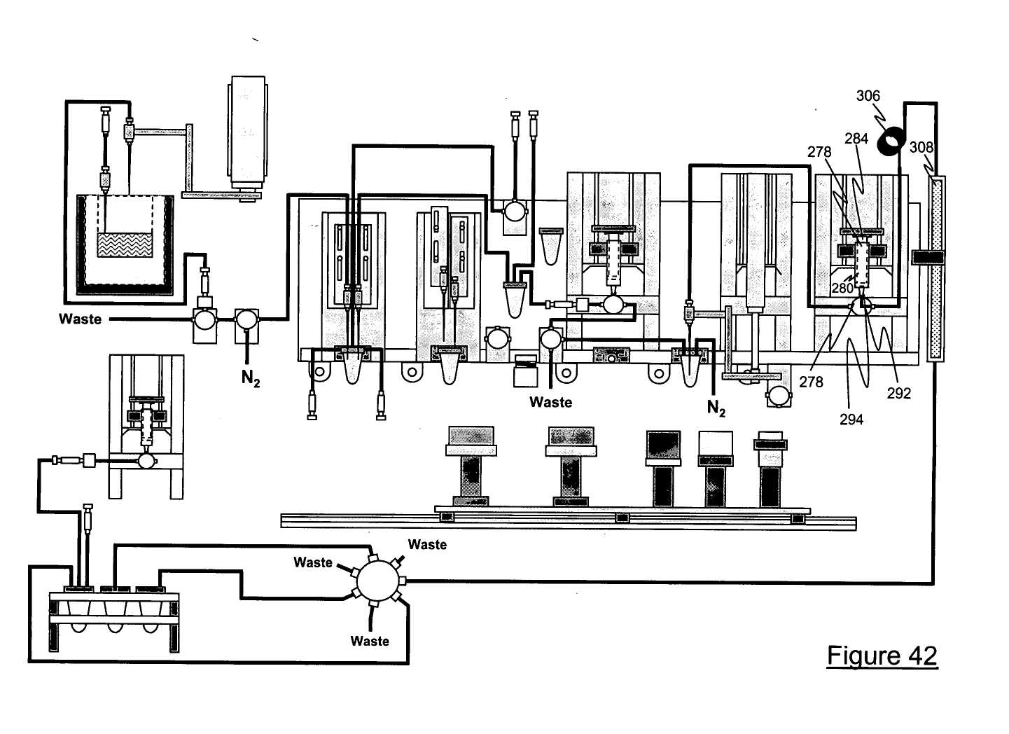

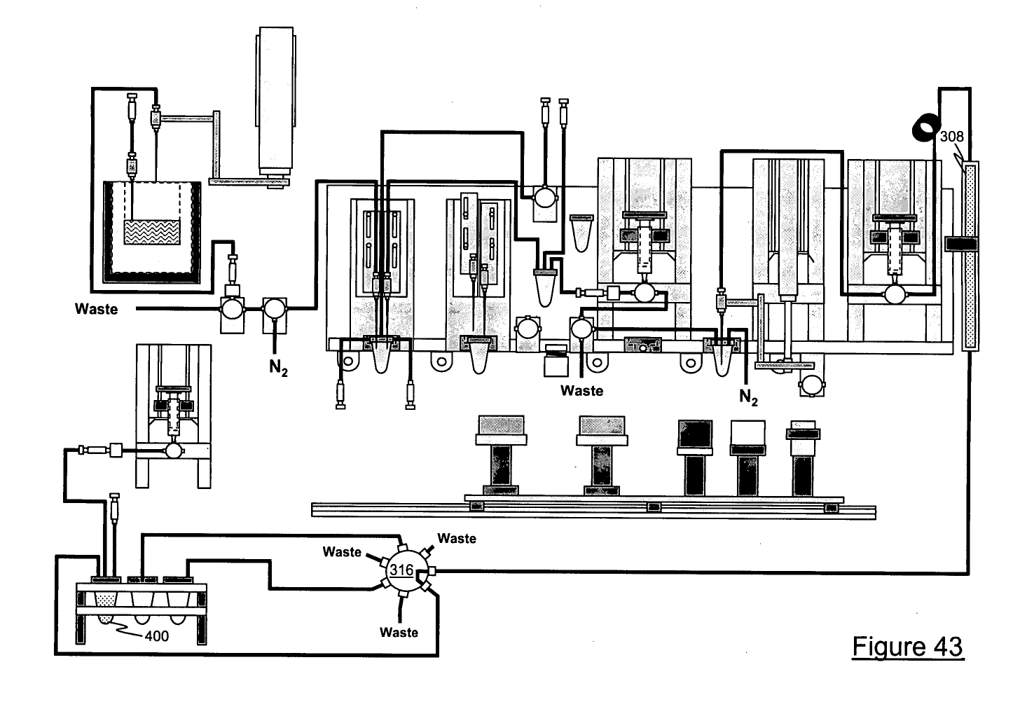

Referring to FIG. 41 and FIG. 42, the mobile phase of the HPLC column 308 may then be introduced into the HPLC column 308. This may be accomplished by introducing the mobile phase via an external input device or via the second syringe device 276 by disposing the mobile phase within the second syringe cavity 280 and configuring the sixth configurable flow direction device 278 such that the sixth valve first port 292 is communicated with the sixth valve second port 294. The second syringe device 276 may then be operated to cause the actuatable plunger portion 284 to traverse the second syringe cavity 280 such that the mobile phase of the HPLC column 308 contained with the second syringe cavity 280 is introduced into the HPLC loop 306. Referring to FIG. 43, the CH₃CN eluent containing the ¹⁸F is ejected from the HPLC column 308 and directed toward the seventh configurable flow direction device 316, wherein the seventh configurable flow direction device 316 may be controllably configured such that the CH₃CN eluent containing the ¹⁸F is directed to a fifth vial 400, wherein the fifth vial 400 defines a fifth vial cavity 402.

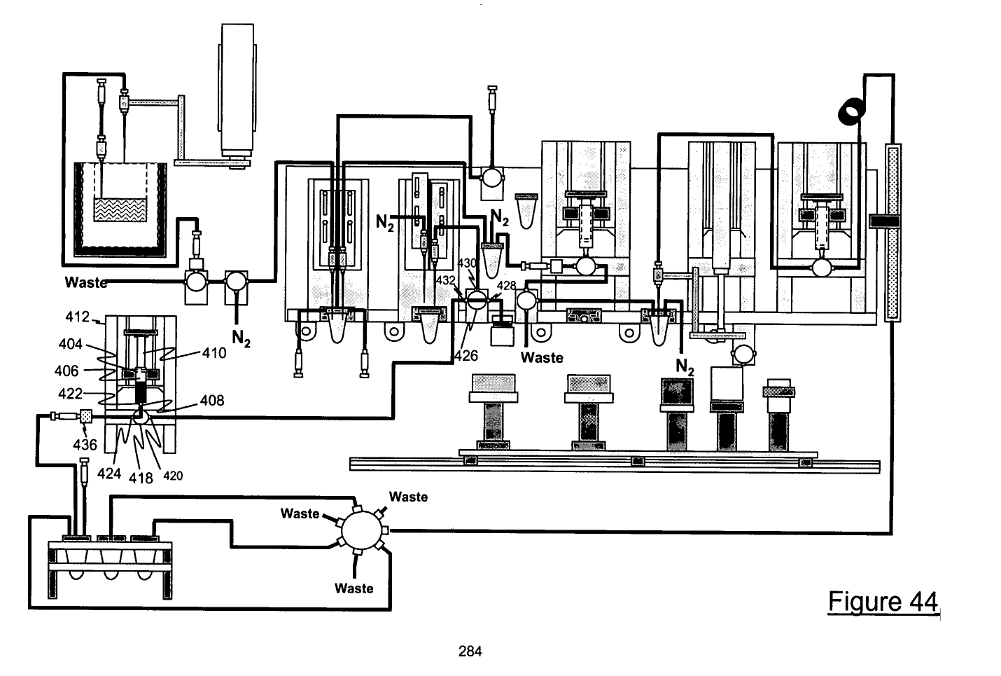

Referring to FIG. 44, the RCSD 100 also includes a third Sep-Pak device 436 and a third syringe device 404, wherein the third syringe device 404 defines a third syringe cavity 406 and includes a third syringe cavity opening 408 and an actuatable plunger portion 410, wherein the actuatable plunger portion 410 is movably disposed within at least a portion of the third syringe cavity 406 and wherein the actuatable plunger portion 410 is controllably configurable between a first plunger configuration 412 and a second plunger configuration 414 via a third plunger actuation device 416. The fifth vial cavity 402 is communicated with the third syringe cavity 406 via a seventh configurable flow direction device 418, wherein the seventh configurable flow direction device 418 includes a seventh valve first port 420, a seventh valve second port 422 and a seventh valve third port 424, wherein the third syringe cavity 406 is communicated with the seventh valve second port 422 and wherein the seventh valve third port 424 is communicated with the fifth vial cavity 402. An eighth configurable flow direction device 426 is also provided and includes an eighth valve first port 428, an eighth valve second port 430 and an eighth valve third port 432, wherein the eighth valve first port 428 is communicated with a product container 434, the eighth valve second port 430 is communicated with the second vial 194 via the fourth needle device 202 and wherein the eighth valve third port 432 is communicated with the seventh valve first port 420. The seventh configurable flow direction device 418 is configured such that the seventh valve second port 422 is communicated with the seventh valve third port 424 and the third syringe device 404 is operated to cause the actuatable plunger portion 410 to traverse the third syringe cavity 406 to draw the CH₃CN eluent containing the ¹⁸F from the fifth vial cavity 402 into the third syringe cavity 406 such that the ¹⁸F compound is retained within the third Sep-Pak device 436 and such that the CH₃CN eluent is contained within the third syringe cavity 406.

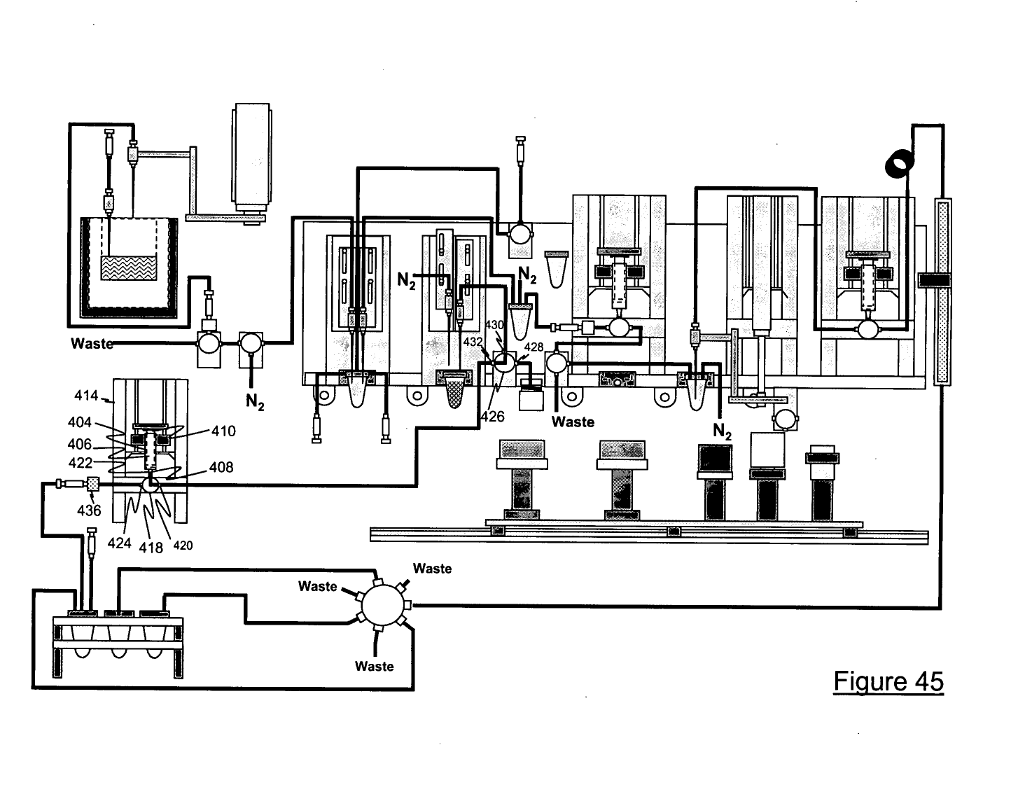

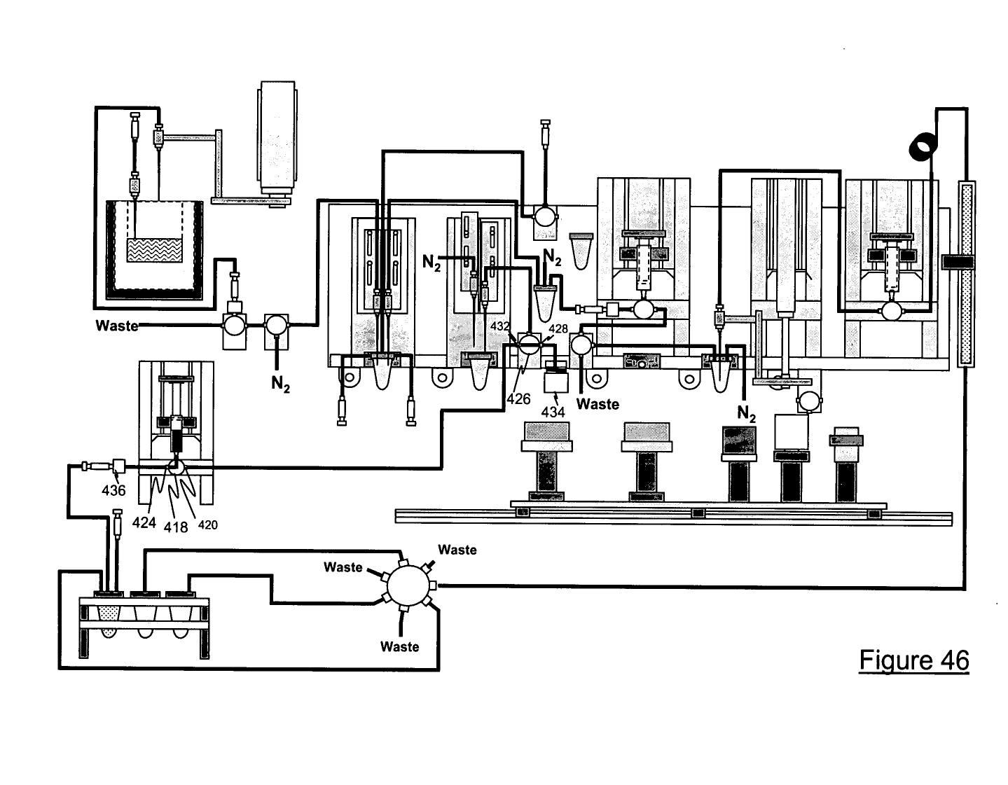

Referring to FIG. 45, the seventh configurable flow direction device 418 is configured such that the seventh valve second port 422 is communicated with the seventh valve first port 420 and eighth configurable flow direction device 426 is configured such that the eighth valve third port 424 is communicated with the eighth valve second port 422. The third syringe device 404 is operated to cause the CH₃CN eluent to flow from the third syringe cavity 406 and into the second vial cavity 196 via the fourth needle device 202. Referring to FIG. 46, the seventh configurable flow direction device 418 is configured such that the seventh valve second port 422 is communicated with the seventh valve third port 424 and a solution may be introduced into the fifth vial cavity 402 via an external input device 193. At this point, the third syringe device 404 may be operated to cause the actuatable plunger portion 410 to traverse the third syringe cavity 406 to draw the solution from the fifth vial cavity 402 through the third Sep-Pak device 436 and into the third syringe cavity 406 such that any ¹⁸F compound retained within the third Sep-Pak device 436 is dislodged and contained within the third syringe cavity 406.

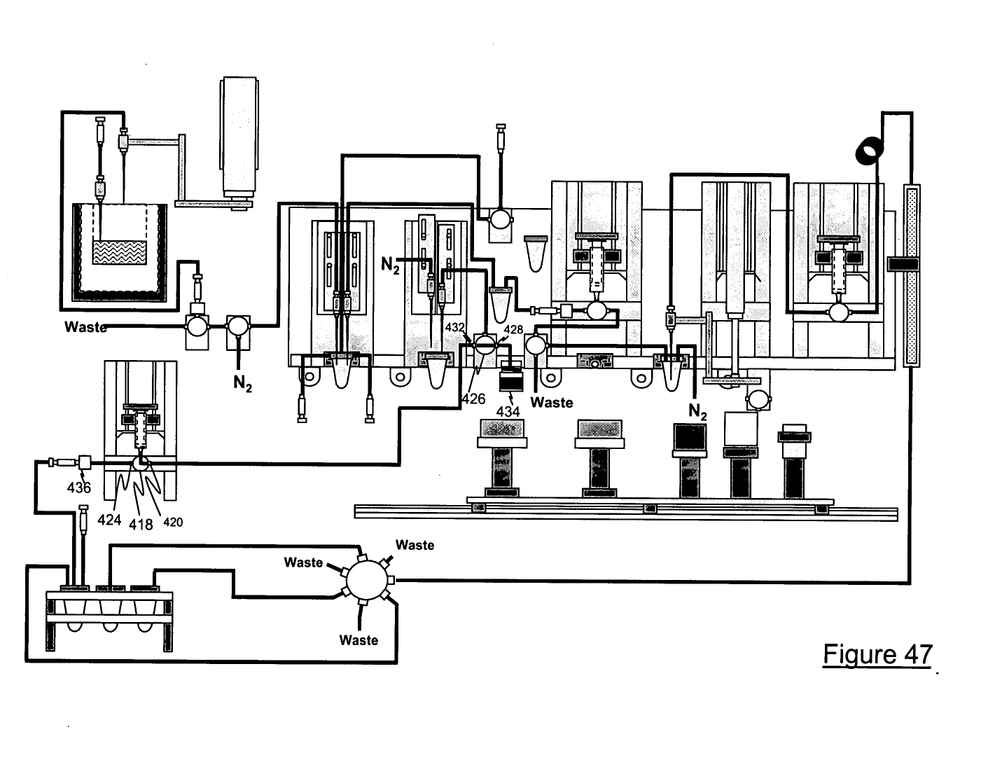

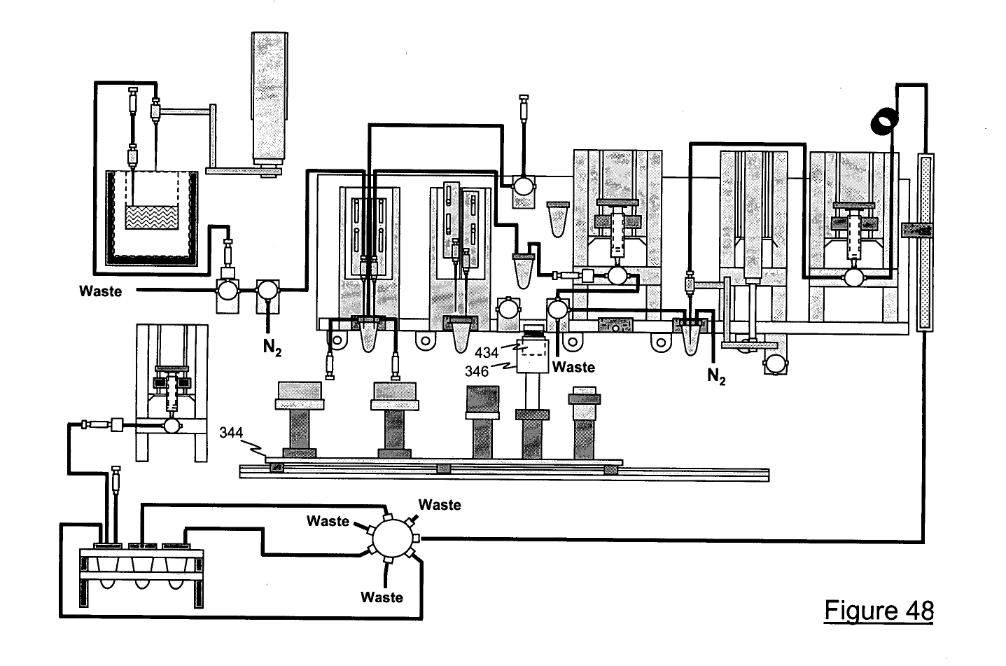

Referring to FIG. 47, the seventh configurable flow direction device 418 is configured such that the seventh valve second port 422 is communicated with the seventh valve first port 420 and the eighth configurable flow direction device 426 is configured such that the eighth valve third port 424 is communicated with the eighth valve first port 428. The third syringe device 404 is operated to cause the solution containing the ¹⁸F compound to flow from the third syringe cavity 406 and into the product container 434. Referring to FIG. 48, the support platform 344 may then be configured such that the shielding structure 346 is disposed directly below the product container 400 and the shielding structure 346 is raised such that the product container 400 is disposed within the shielding structure 346. The product container 434 may then be removed from the RCSD 100 for later processing, as shown in operational block 512.

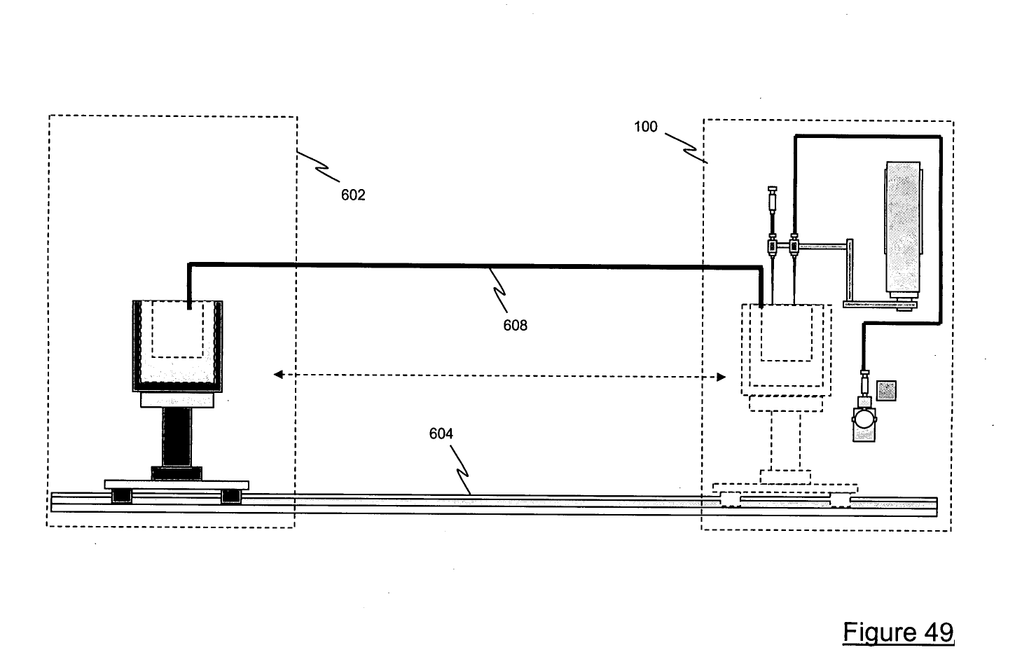

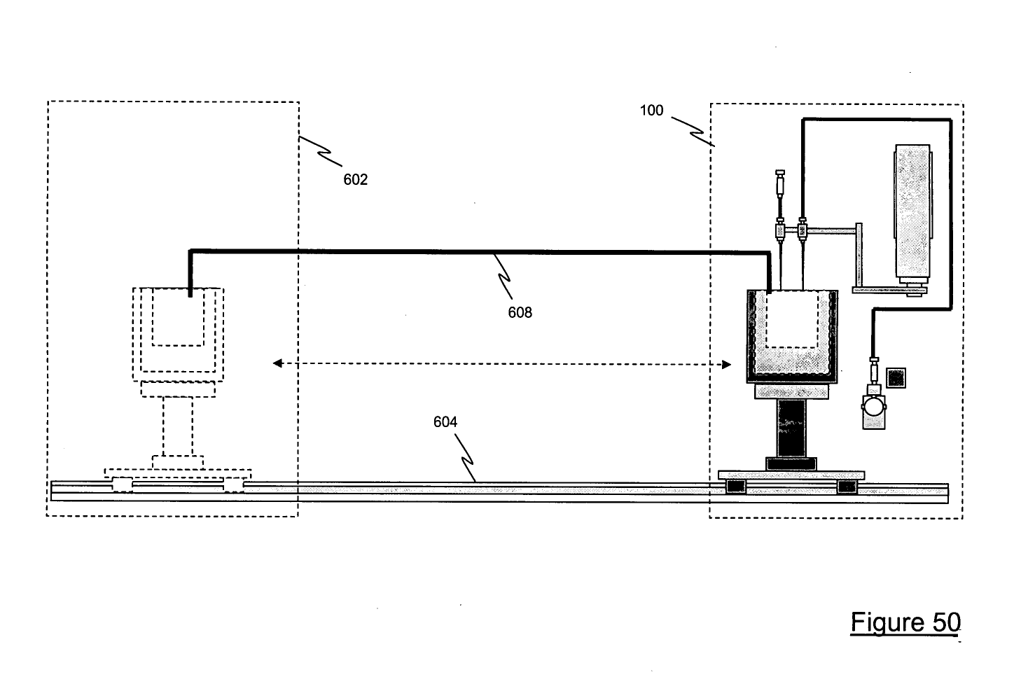

Referring to FIG. 49 and FIG. 50, an additional embodiment may allow the RCSD 100 to be associated with a preprocessing device, such as a cyclotron 602, wherein the output of the cyclotron 602 may be connected to a container within the RCSD 100 to allow a hazardous and/or radioactive substance to be transferred directly from the cyclotron 602 to the RCSD 100 via an automated process that may be operated via the RCSD 100 and/or via a remotely operated initial substance transfer device 604, wherein the remotely operated initial substance transfer device may be at least one of a shielded container conveyor system 606 and a transfer flow tube 608. This type of set up would not only decrease the risk of radiation exposure to the radiochemist to almost zero (0), it would also be beneficial when working with substances that have a short half life, such as Carbon-11 (¹¹C) which has a half life of approximately 20 minutes. This is because the substance can be introduced into the synthesis process directly out of the cyclotron 602. Furthermore, the first radiation monitoring device 192 and the second radiation monitoring device 216 may be positionably configurable to monitor the radioactivity of the initial substance and the processed substance in at least one of the first synthesis portion 102 and the second synthesis portion 104. Similarly, the first temperature sensing device 190 and the second temperature sensing device 214 may be positionably configurable to monitor the temperature of at least one of the first vial 156, the second vial 194, the third vial 218, the fourth vial 268, the first syringe device 244, the second syringe device 276, the first Sep-Pak device 138, the second Sep-Pak device 222 and the at least one device structure, wherein the at least one device structure includes at least one of a first bathing structure 334, a second bathing structure 338, a third bathing structure 342, a shielding structure 346 and a microwave device 350.

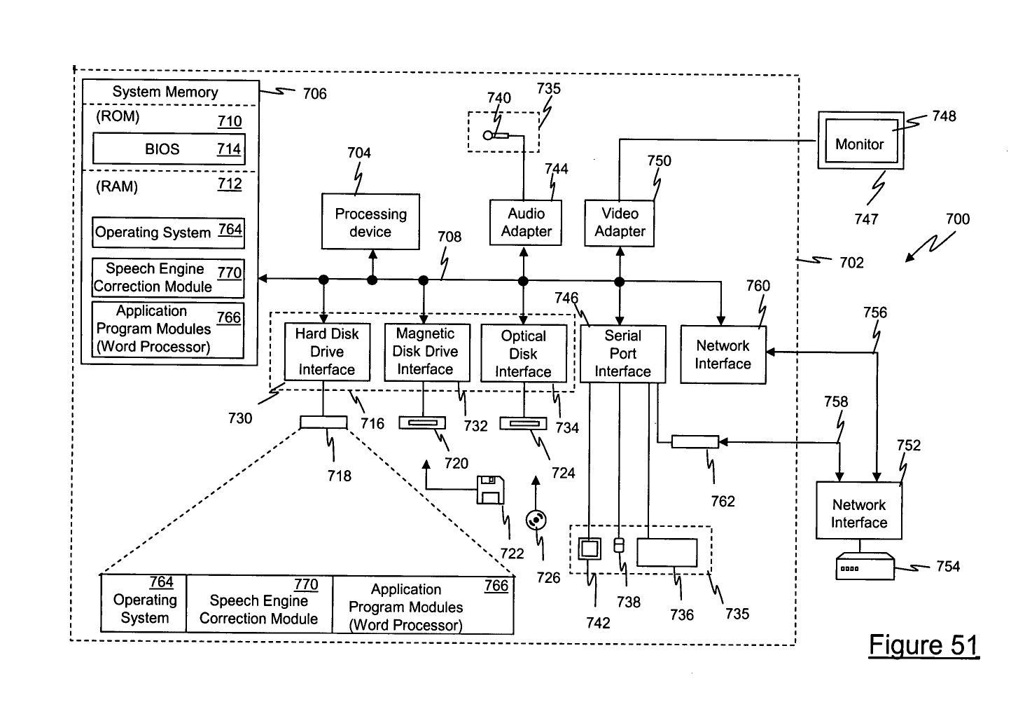

Referring to FIG. 51, a block diagram illustrating a system 700 for operating the RCSD 100 via a software User Interface (UI) window on a display screen is shown and includes a general computer system 702, having a processing device 704, a system memory 706, and a system bus 708, wherein the system bus 708 couples the system memory 706 to the processing device 704. The system memory 706 may include read only memory (ROM) 710 and random access memory (RAM) 712. A basic input/output system 714 (BIOS), containing basic routines that help to transfer information between elements within the general computer system 702, such as during start-up, may be stored in ROM 710. The general computer system 702 further includes a storage device 716, such as a hard disk drive 718, a magnetic disk drive 720, e.g., to read from or write to a removable magnetic disk 722, and an optical disk drive 724, e.g., for reading a CD-ROM disk 726 or to read from or write to other optical media. The storage device 716 may be connected to the system bus 708 by a storage device interface, such as a hard disk drive interface 730, a magnetic disk drive interface 732 and an optical drive interface 734. The drives and their associated computer-readable media provide nonvolatile storage for the general computer system 702. Although the description of computer-readable media above refers to a hard disk, a removable magnetic disk and a CD-ROM disk, it should be appreciated that other types of media that are readable by a computer system and that are suitable to the desired end purpose may be used, such as magnetic cassettes, flash memory cards, digital video disks, Bernoulli cartridges, and the like.

A user may enter commands and information into the general computer system 702 through a conventional input device 735, including a keyboard 736, a pointing device, such as a mouse 738 and a microphone 740, wherein the microphone 740 may be used to enter audio input, such as speech, into the general computer system 702. Additionally, a user may enter graphical information, such as drawings or hand writing, into the general computer system 702 by drawing the graphical information on a writing tablet 742 using a stylus. The general computer system 702 may also include additional input devices suitable to the desired end purpose, such as a joystick, game pad, satellite dish, scanner, or the like. The microphone 740 may be connected to the processing device 704 through an audio adapter 744 that is coupled to the system bus 708. Moreover, the other input devices are often connected to the processing device 704 through a serial port interface 746 that is coupled to the system bus 708, but may also be connected by other interfaces, such as a game port or a universal serial bus (USB).

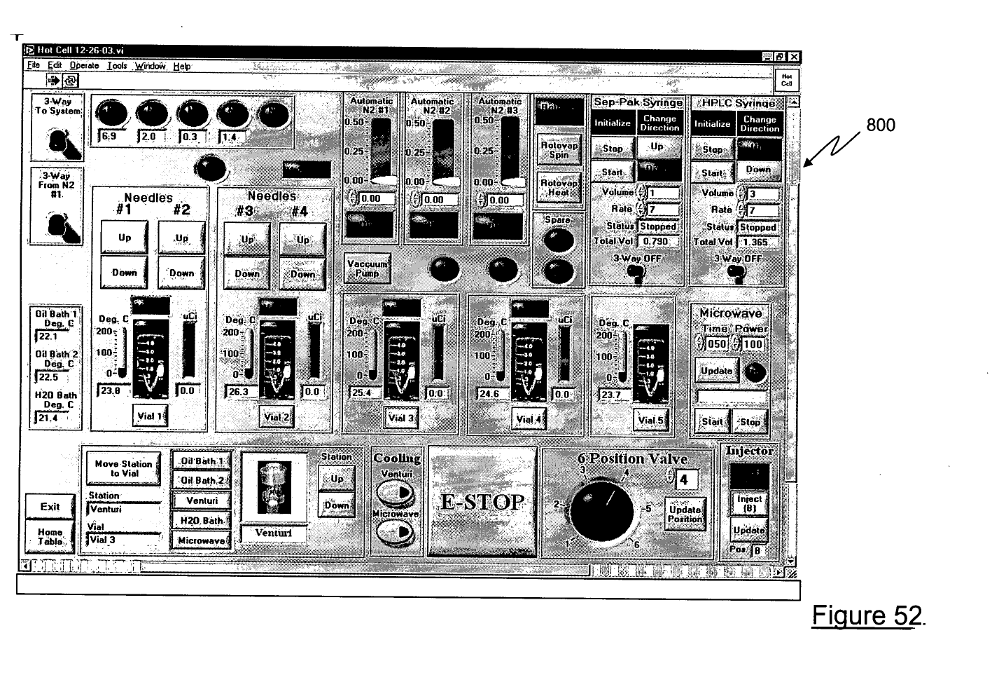

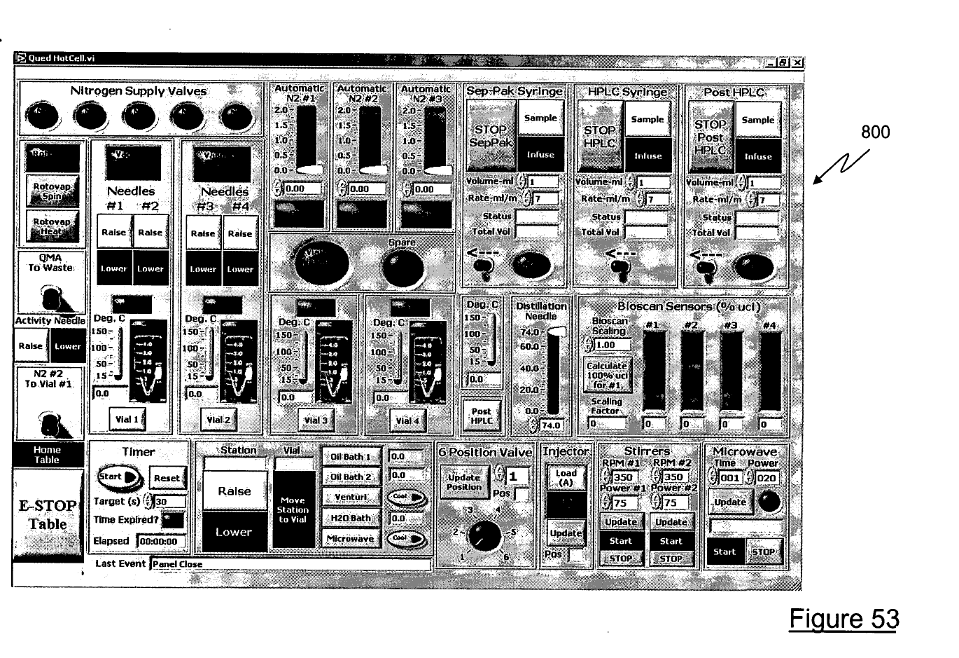

A display device 747, such as a monitor or other type of display device 747, having a display screen 748, is also connected to the system bus 708 via an interface, such as a video adapter 750. In addition to the display screen 748, the general computer system 702 may also typically include other peripheral output devices, such as speakers and/or printers. The general computer system 702 may operate in a networked environment using logical connections to one or more remote computer systems 752. The remote computer system 752 may be a server, a router, a peer device or other common network node, and may include any or all of the elements described relative to the general computer system 702, although only a remote memory storage device 754 has been illustrated in FIG. 51. The logical connections as shown in FIG. 51 include a local area network (LAN) 756 and a wide area network (WAN) 758. Such networking environments are commonplace in offices, enterprise-wide computer networks, intranets and the Internet. Referring to FIG. 52 and FIG. 53, a couple of different embodiments of a Software Graphical User Interface 800 are shown wherein each includes software controls for controlling each of the operable elements in the RCSD 100.

When used in a LAN networking environment, the general computer system 702 is connected to the LAN 756 through a network interface 760. When used in a WAN networking environment, the general computer system 702 typically includes a modem 762 or other means for establishing communications over a WAN 758, such as the Internet. The modem 762, which may be internal or external, may be connected to the system bus 708 via the serial port interface 746. In a networked environment, program modules depicted relative to the general computer system 702, or portions thereof, may be stored in the remote memory storage device 754. It should be appreciated that the network connections shown are exemplary and other means of establishing a communications link between the computer systems may be used. It should also be appreciated that the application module could equivalently be implemented on host or server computer systems other than general computer systems, and could equivalently be transmitted to the host computer system by means other than a CD-ROM, for example, by way of the network connection interface 760. Furthermore, a number of program modules may be stored in the drives and RAM 712 of the general computer system 702. Program modules may control how the general computer system 702 functions and interacts with the user, with I/O devices or with other computers. Program modules may include routines, operating systems 764, target application program modules 766, data structures, browsers, and other software or firmware components. The method 500 of FIG. 27 may be included in an application module and the application module may conveniently be implemented in one or more program modules, such as a multi-system RSCD 770 based upon the method(s) described herein. The target application program modules 766 may comprise a variety of applications used in conjunction with the method 500 and/or the RCSD 100.

It should be appreciated that no particular programming language is described for carrying out the various procedures described in the detailed description because it is considered that the operations, steps, and procedures described and illustrated in the accompanying drawings are sufficiently disclosed to permit one of ordinary skill in the art to practice an exemplary embodiment of the present invention. Moreover, there are many computers and operating systems which may be used in practicing an exemplary embodiment, and therefore no detailed computer program could be provided which would be applicable to all of these many different systems. Each user of a particular computer should be aware of the language and tools which are most useful for that user's needs and purposes. It should also be appreciated that although the embodiment disclosed herein is disclosed with reference to a process involving an ¹⁸F compound, the embodiment may be configurable to accommodate any substance and/or process involving a fluid, a solid and/or a gas and each of the elements contained herein may be interchanged and configurable, either manually and/or automatically, to interact with any of the other elements contained herein.

Moreover, at least one of the input needle actuation device 122, the first needle actuation device 172, the second needle actuation device 174, the third needle actuation device 210, the fourth needle actuation device 212, the plunger actuation device 254, the second plunger actuation device 290, the fifth needle actuation device 304, the first bathing structure actuation device 358, the second bathing structure actuation device 360, the third bathing structure actuation device 362, the shielding structure actuation device 364, the microwave structure actuation device 366 and the support platform actuation device 370 may be any type of actuation device suitable to the desired end purpose, such as a pneumatic actuation device, a mechanical actuation device, an electrical actuation device and/or any combination thereof. It should also be appreciated that the transfer of fluid between vials and/or needles may be accomplished via pressure generated by any pressure generation device suitable to the desired end purpose, such as an N₂ generation device. Furthermore, although the syringe devices 226, 276, 404 are shown as being Harvard Syringe pumps, any type of syringe devices 226, 276, 404 suitable to the desired end purpose may be used.

All or some of the vials and/or containers used within the RCSD 100 may include a self sealing membrane to contain any material within the vials and/or containers, wherein the membrane would be punctured with the needles to remove and/or introduce a substance into the vials and/or containers. As such, with the use of the self sealing membranes the RCSD 100 may be used for synthesis processes that include all forms of matter, including liquid and gaseous substances. Additionally, although the embodiment of the remote-controlled synthesis device (RCSD) 100 as described hereinabove has been described in terms of an RCSD 100 configured for synthesizing and purifying an ¹⁸F compound, it should be appreciated that the RCSD 100 may be configured into any configuration suitable to the desired end purpose to conduct any synthesis process suitable to the desired end purpose, such as the radiosynthesis of Carbon-11 (¹¹C). For example, the RCSD 100 may be configured to include a plurality of input stations, synthesis portions and/or other type of processing stations, such as HPLC stations beyond what is illustrated herein. As such, the RCSD 100 may include single and/or multiple stations/portions as desired. Alternatively, the RCSD 100 may be configured to include more or less switching devices and/or flow direction devices than what is illustrated herein. It is further contemplated that each of the elements of the RCSD 100 may be controllably configurable individually and/or as a system, suitable to the desired end purpose.

Moreover, it should be appreciated although the method 500 for implementing the remote-controlled synthesis device 100 is described herein in terms of a typical ¹⁸F synthesis and purification process, the method 500 and the remote-controlled synthesis device 100 may be applied to any synthesis process suitable to the desired end purpose, such as the radiosynthesis of Carbon-11 (¹¹C). Furthermore, the remote-controlled synthesis device (RCSD) 100 may also be used for synthesis involving substances in gaseous form, such as Deuterium and Tritium (T-2). Also, although as disclosed herein the compound under study contained within the vials are shown as being moved at a predetermined flow rate (such as 10 mils/minute) via N₂ pressure and/or via suction generated from a syringe device, it is contemplated that the compound may be moved via any method and/or device suitable to the desired end purpose.

Referring to FIG. 51, the method of FIG. 27 may be implemented through a processing device operating in response to a computer program. In order to perform the prescribed functions and desired processing, as well as the computations therefore (e.g., the execution of fourier analysis algorithm(s), the control processes prescribed herein, and the like), the controller may include, but not be limited to, a processor(s), computer(s), memory, storage, register(s), timing, interrupt(s), communication interfaces, and input/output signal interfaces, as well as combinations comprising at least one of the foregoing. For example, the controller may include signal input signal filtering to enable accurate sampling and conversion or acquisitions of such signals from communications interfaces. It is considered within the scope of the invention that the processing of FIG. 27 may be implemented by a controller located remotely from the processing device. Furthermore, the processing device may be operated to control the Remote Controlled Synthesis Device 100 according to a predetermined script or the processing device may be operated via real time input from a radiochemist instructing the Remote Controlled Synthesis Device 100 via a Graphic User Interface (GUI) as that shown in FIG. 52 and FIG. 53. This allows the Remote Controlled Synthesis Device 100 to be used for a broad range of applications, from a simple synthesis application to an extremely complicated and dynamic synthesis application.

Moreover, the method of FIG. 27 may be embodied in the form of computer-implemented processes and apparatuses for practicing those processes. The above may also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Existing systems having reprogrammable storage (e.g., flash memory) can be updated to implement the invention. The above can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments may configure the microprocessor to create specific logic circuits in whole or in part.

While the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

We claim:

1. A synthesis system, comprising:
a first synthesis portion, wherein said first synthesis portion includes,
a first station, wherein said first station includes an input needle communicated with a first Sep-Pak device via a first flow tube, wherein said first Sep-Pak device is connected to at least one configurable flow direction device which is further communicated with a first needle, said first needle being configurable to be at least partially disposed within a first vial cavity defined by a first vial, said first station further including a second needle, wherein said second needle is configurable to be at least partially disposed within said first vial cavity,
a second station, wherein said second station includes a second vial defining a second vial cavity, a third needle and a fourth needle, wherein said third needle and said fourth needle are configurable to be at least partially disposed within said second vial cavity,
a third station, wherein said third station includes a third vial defining a third vial cavity, wherein said third vial cavity is communicated with said second needle and a second Sep-Pak device, wherein said second Sep-Pak device is further communicated with at least one of a first syringe device and a fourth vial cavity defined by a fourth vial via said at least one configurable flow direction device,
a fourth station, wherein said fourth station includes a fifth needle configurable to be at least partially disposed within said fourth vial cavity, wherein said fifth needle is communicated with at least one of a second syringe device and an HPLC loop via said at least one configurable flow direction device, and
an HPLC station, wherein said HPLC station includes an HPLC column having an HPLC input port and an HPLC output port, said HPLC input port communicated with said HPLC loop and said HPLC output port communicated with said at least one configurable flow device; and
a second synthesis portion, wherein said second synthesis portion includes,
a support platform and at least one device structure, wherein said support platform is configurable to dispose said at least one device structure adjacent at least one of said first vial, said second vial and said third vial and wherein said at least one device structure is configurable to be associated with at least one of said first vial, said second vial and said third vial.

2. The synthesis system of claim 1, wherein said at least one configurable flow direction device includes at least one of a first configurable flow direction device, a second configurable flow direction device, a third configurable flow direction device, a fourth configurable flow direction device and a fifth configurable flow device.

3. The synthesis system of claim 2, wherein said first configurable flow direction device is communicated with said first Sep-Pak device, at least one of an N₂ supply device and a waste container and said second configurable flow direction device and wherein said second configurable flow direction device is further communicated with an N₂ supply device and a first needle.

4. The synthesis system of claim 1, further comprising at least one external input device communicated with at least one of said at least one configurable flow direction device, said first vial, said second vial, said third vial and said fourth vial.

5. The synthesis system of claim 1, wherein at least one of said input needle, said first needle, said second needle, said third needle, said fourth needle and said fifth needle are configured via at least one controllable needle actuation device.

6. The synthesis system of claim 1, further comprising a shielded input container defining a container cavity for containing a product, wherein at least one of said shielded input container and said input needle is configurable such that said input needle is at least partially disposed within said container cavity.

7. The synthesis system of claim 1, wherein said first syringe device defines a first syringe cavity and includes a first syringe plunger, wherein said first syringe plunger is movably associated with said first syringe device to be at least partially disposed within said first syringe cavity such that said first syringe plunger traverses the length of said first syringe cavity.

8. The synthesis system of claim 1, wherein said second syringe device defines a second syringe cavity and includes a second syringe plunger, wherein said second syringe plunger is movably associated with said second syringe device to be at least partially disposed within said second syringe cavity such that said second syringe plunger traverses the length of said second syringe cavity.

9. The synthesis system of claim 1, further comprising at least one syringe actuation device, wherein said at least one syringe actuation device is associated with at least one of said first syringe device and said second syringe device to configure said at least one of said first syringe device and said second syringe device between an engaged configuration and a disengaged configuration.

10. The synthesis system of claim 1, further comprising at least one syringe actuation device, at least one needle actuation device, at least one N₂ supply device, at least one vacuum device and at least one configurable flow direction device, each of which are separately controllable via a processing device.

11. The synthesis system of claim 1, wherein said at least one configurable flow direction device includes at least one three-way directional flow device and at least one seven-way flow direction device, wherein said at least one three-way directional flow device includes three configurable ports and wherein said at least one seven-way directional flow device includes seven configurable ports.

12. The synthesis system of claim 1, wherein said support platform is movably associated with the second synthesis portion such that said support platform is movable in at least one of an xy-plane, an xz-plane, a yz-plane and an xyz-plane.

13. The synthesis system of claim 1, wherein at least a portion of said at least one device structure is movably associated with said support platform in at least one of an xy-plane, an xz-plane, a yz-plane and an xyz-plane.

14. The synthesis system of claim 1, wherein said at least one device structure includes at least one of a first bathing device having a first bathing structure, a second bathing device having a second bathing structure, a third bathing device having a third bathing structure, a shielding device having a shielding structure and a microwave device having a microwave structure.

15. The synthesis system of claim 1, wherein at least one of said first bathing structure, said second bathing structure, said third bathing structure, said shielding structure and said microwave structure is configurable between an extended configuration and a contracted configuration, wherein when said at least one of said first bathing structure, said second bathing structure, said third bathing structure, said shielding structure and said microwave structure are configured in said extended configuration said at least one of said first bathing structure, said second bathing structure, said third bathing structure, said shielding structure and said microwave structure is associated with at least one of said first station, said second station, said third station and said fourth station.

16. The synthesis system of claim 1, further comprising at least one support structure actuation device and at least one device structure actuation device, each of which is separately controllable via a processing device.

17. The synthesis system of Clam 1, further comprising a system enclosure, wherein said system enclosure defines an enclosure cavity for at least partially containing the synthesis system and a plurality of openings disposed to allow access to the synthesis system.

18. The synthesis system of claim 1, further comprising at least one of a radiation monitoring device and a UV monitoring device, wherein said at least one radiation monitoring device and said UV monitoring device is positionably configurable to monitor the radioactivity of said initial substance and said processed substance in at least one of said first synthesis portion and said second synthesis portion.

19. The synthesis system of claim 1, further comprising at least one temperature monitoring device, wherein said at least one temperature monitoring device is positionably configurable to monitor the temperature of at least one of said first vial, said second vial, said third vial and said fourth vial, said first syringe device, said second syringe device, said first Sep-Pak device, said second Sep-Pak device and said at least one device structure.

20. The synthesis system of claim 1, further comprising at least one flow actuation device, said at least one flow actuation device being communicated with at least one of said first vial, said second vial, said third vial and said fourth vial, said first syringe device, said second syringe device, said first Sep-Pak device, said second Sep-Pak device and said HPLC station.

21. A synthesis system, comprising:
at least one synthesis portion, wherein said at least one synthesis portion includes at least one synthesis portion input device;
at least one processing portion, wherein said at least one processing portion includes at least one processing portion input device; and
at least one support device for securably supporting a container, wherein said at least one support device is configurable to support a plurality of different sized and shaped containers and wherein said at least one support device is communicated with at least one of said at least one synthesis portion and said at least one processing portion via at least one configurable flow valve.

22. The synthesis system of claim 21, wherein said at least one configurable flow valve is communicated with said at least one synthesis portion and said at least one processing portion via a flow tube.

23. The synthesis system of claim 21, wherein at least one of said at least one synthesis portion, said at least one processing portion, said at least one support device and said at least one configurable flow valve is controllably configurable via a remote device.

24. The synthesis system of claim 21, wherein said at least one synthesis portion includes a plurality of synthesis portions.

25. The synthesis system of claim 21, wherein said at least one processing portion includes a plurality of processing portions.

26. The synthesis system of claim 21, wherein said at least one support device includes a plurality of support devices.

27. The synthesis system of claim 21, wherein said at least one configurable flow valve includes a plurality of configurable flow valves.

28. A synthesis system, comprising:
a first synthesis portion, wherein said first synthesis portion includes,
at least one input device, at least one material collection device, at least one container and at least one configurable flow direction device; and
a second synthesis portion, wherein said second synthesis portion includes,
a support platform and at least one substance processing device structure, wherein at least one of said support platform and said at least one substance processing device structure is configurable to dispose said at least one substance processing device structure adjacent said at least one container.

29. A method for implementing a synthesis system having a first synthesis portion and a second synthesis portion, wherein the first synthesis portion includes a plurality of synthesis stations having at least one input device, at least one output device and at least one container defining a container cavity, wherein each of the at least one input and the at least one output is configurably communicated with the container cavity and wherein the second synthesis portion includes a support platform and at least one substance processing device structure, wherein at least one of the support platform and the at least one substance processing device structure is configurable to dispose the at least one substance processing device structure adjacent at least one of the plurality of synthesis stations wherein the at least one substance processing device structure is configurable to be associated with the at least one container, the method comprising:
arranging at least one of the plurality of synthesis stations in a predetermined configuration, wherein said predetermined configuration is responsive to an initial substance to be processed;
introducing said initial substance into said at least one of the plurality of synthesis stations via the at least one input;
operating the synthesis system to process said initial substance responsive to a predetermined algorithm to generate a processed substance; and
collecting said processed substance.

30. The method of claim 29, wherein the plurality of synthesis stations includes a first synthesis station, a second synthesis station, a third synthesis station, a fourth synthesis station and an HPLC station.

31. The method of claim 30, wherein said at least one substance processing device structure includes at least one of a first bathing device having a first bathing structure, a second bathing device having a second bathing structure, a third bathing device having a third bathing structure, a shielding device having a shielding structure and a microwave device having a microwave structure.

32. The method of claim 31, wherein said arranging includes communicating at least one of said first synthesis station with at least one of said second synthesis station, said third synthesis station, said fourth synthesis station and said HPLC station.

33. The method of claim 29, wherein said introducing includes operating the synthesis system to cause a substance to be introduced into at least one of said plurality of synthesis stations via the at least one input.

34. The method of claim 29, wherein said introducing includes introducing said initial substance into the synthesis system via an automated process.

35. The method of claim 34, wherein said automated process includes directly transferring said initial substance between a preprocessing device and the synthesis system via a remotely operated initial substance transfer device.

36. The method of claim 35, wherein said preprocessing device is a cyclotron and wherein said remotely operated initial substance transfer device is at least one of a shielded container conveyor system and a transfer flow tube.

37. The method of claim 29, wherein said introducing includes causing said substance to be introduced into at least one of said plurality of synthesis stations via pneumatic pressure generated via a pneumatic fluid.

38. The method of claim 37, wherein said pneumatic fluid is N₂.

39. The method of claim 31, wherein said operating includes associating at least one of said first bathing device, said second bathing device, said third bathing device, said shielding device and said microwave device with at least one of said first synthesis station, said second synthesis station, said third synthesis station, said fourth synthesis station and said HPLC station.

40. The method of claim 29, wherein said operating further includes operating the synthesis system via a Graphical User Interface wherein said Graphical User Interface is communicated with a processing device.

41. The method of claim 29, wherein said operating includes introducing additional substances into at least one of said plurality of synthesis stations via the at least one input.

42. The method of claim 41, wherein said the at least one input is at least one of a manual input device and an automated input device.

43. The method of claim 29, wherein said collecting includes operating the synthesis system to dispose said processed substance within a shield container.

44. A machine-readable computer program code, the program code including instructions for causing a controller to implement a method for implementing a synthesis system having a first synthesis portion and a second synthesis portion, wherein the first synthesis portion includes a plurality of synthesis stations having at least one input device, at least one output device and at least one container defining a container cavity, wherein each of the at least one input and the at least one output is configurably communicated with the container cavity and wherein the second synthesis portion includes a support platform and at least one substance processing device structure, wherein at least one of the support platform and the at least one substance processing device structure is configurable to dispose the at least one substance processing device structure adjacent at least one of the plurality of synthesis stations wherein the at least one substance processing device structure is configurable to be associated with the at least one container, the method comprising:
arranging at least one of the plurality of synthesis stations in a predetermined configuration, wherein said predetermined configuration is responsive to an initial substance to be processed;
introducing said initial substance into said at least one of the plurality of synthesis stations via the at least one input;
operating the synthesis system to process said initial substance responsive to a predetermined algorithm to generate a processed substance; and
collecting said processed substance.

45. The machine-readable computer program code of claim 44, wherein said machine-readable computer program code is encoded onto a storage medium.
